(19) United States
(12) Patent Application Publication
(10) Pub. No.: US 2015/0106975 A1
(43) Pub. Date: Apr. 16, 2015

(54) Molecular Markers Associated with Aphid Resistance in Soybean

(71) Applicants: Vergel C. Concibido, St. Louis, MO (US); Susannah G. Cooper, St. Louis, MO (US); Katy Hillard, St. Louis, MO (US); David Hoffman, St. Louis, MO (US); Ryan Rapp, St. Louis, MO (US); Dennis Yang, St. Louis, MO (US); Jennifer Yates, St. Louis, MO (US)

(72) Inventors: Vergel C. Concibido, St. Louis, MO (US); Susannah G. Cooper, St. Louis, MO (US); Katy Hillard, St. Louis, MO (US); David Hoffman, St. Louis, MO (US); Ryan Rapp, St. Louis, MO (US); Dennis Yang, St. Louis, MO (US); Jennifer Yates, St. Louis, MO (US)

(73) Assignee: Monsanto Technology LLC, St. Louis, MO (US)

(21) Appl. No.: 14/241,282

(22) PCT Filed: Aug. 29, 2012
(86) PCT No.: PCT/US12/52891

§ 371 Date: Nov. 24, 2014

Related U.S. Application Data
(60) Provisional application No. 61/529,879, filed on Aug. 31, 2011.

(51) Int. Cl. C12Q 1/68; C12N 15/82

(52) U.S. Cl. CPC C12Q 1/6895; C12N 15/8286; C12Q 2600/158; C12Q 2600/13; C12Q 2600/156
USPC 800/302; 435/6.11

(57) ABSTRACT

The present invention provides methods and compositions for the identification and selection of loci modulating phenotypic expression of an aphid resistance trait in plant breeding. In addition, methods are provided for screening germplasm entries for the performance and expression of this trait.

### CROSS-REFERENCE TO RELATED APPLICATIONS

This international application claims the benefit of U.S. Provisional Patent Application 61/529,879, filed Aug. 31, 2011 and incorporated herein by reference in its entirety.

### INCORPORATION OF SEQUENCE LISTING

A sequence listing containing the file named “46_{—}21_{—}57834. txt” which is 31,766 bytes (measured in MS-Windows®) and created on Aug. 29, 2012, comprises 82 nucleotide sequences, is provided herewith via the USPTO's EFS system and is herein incorporated by reference in its entirety.

### BACKGROUND OF INVENTION

Soybean, Glycine max (L.) Merril, is a major economic crop worldwide and is a primary source of vegetable oil and protein (Sinclair and Backman, Compendium of Soybean Diseases, 3^{rd} Ed. APS Press, St. Paul, Minn., p. 106. (1989). The growing demand for low cholesterol and high fiber diets has also increased soybean's importance as a health food.

Soybean varieties grown in the United States have a narrow genetic base. Six introductions, ‘Mandarin,’ ‘Manchu’, ‘Mandarin’ (Ottawa), “Richland,’ ‘AK’ (Harrow), and ‘Mukden,’ contributed nearly 70% of the germplasm represented in 136 cultivar releases. To date, modern day cultivars can be traced back from these six soybean strains from China. In a study conducted by Cox et al., Crop Sci. 25:529-532 (1988), the soybean germplasm is comprised of 90% adapted materials, 9% unadapted, and only 1% from exotic species. The genetic base of cultivated soybean could be widened through exotic species. In addition, exotic species may possess such key traits as disease, stress, and insect resistance.

Soybean aphid, Aphis glycines Matsumura, was identified as new insect pest of soybeans in 2001 and spread to over 21 states in the United States and 3 Canadian provinces by 2003 (Vennette et al. Ann Entomol Soc Am 97:217-226 (2004)). High yields are critical to a farmer's profit margin. Soybean aphid can cause over 50% yield losses (Wang et al., Plant Protect 20:12-13 (1994)). In addition to the decrease in yield, an increase in insecticide use can also decrease a farmer's profit margin. Over 7 million acres of soybean in the North Central U.S. were sprayed with insecticide to control soybean aphids in 2003; the estimated cost of the insecticide treatments was $84-$105 million in the North Central region alone in 2003 (Landis et al. NCR-125 Arthropod biological control: state reports for 2003; Li et al., Mol Breeding 19:25-34 (2007)).

Soybean aphids can directly damage the plant by removing significant amounts of water and nutrients causing the leaves to yellow and wilt. Additionally, aphids excrete honeydew, a sugar-rich sticky substance, on to the leaves and plants. Honeydew often leads to the development of sooty mold, which affects photosynthesis resulting significant yield losses (Gomez et al., Environ Exp Bot 55: 77-86 (2006). Soybean aphids vector a number of viruses that can stunt plant growth, distorts leaves, cause mottling of leaves and stem, reduce pod number and cause discoloration in the seed. Viruses transmitted via soybean aphid include, Soybean mosaic virus, yellow mosaic virus, tobacco etch virus and tobacco vein mottling virus (Wang et al. Plant Dis 90: 920-926 (2006).

Host plant resistance to insect are often quantitatively inherited traits and not major resistance gene. Stacking quantitative resistances is more durable than a major gene for resistance, but is difficult to identify and incorporate multiple quantitative resistances into a single soybean variety. Molecular markers associated with insect resistance offers breeders a more efficient method to work with quantitative traits and insect resistance. Certain aphid resistance genes and QTLs in soybean are known. Examples of aphid resistance genes and QTLs which including Rag1 were identified in the soybean variety Dowling and mapped to linkage group M (US Patent Application Publication No. 20060015964). Additionally, quantitative trait loci associated with aphid resistance were identified in Plant Introduction (PI) 567598B and mapped linkage groups B2, D1b, J and K (U.S. Pat. No. 7,781,648).

U.S. Pat. No. 7,781,648 further disclosed linkage group J quantitative trait loci associated with aphid resistance identified in soybean Plant Introduction (PI) 567598B that were associated with the markers Sat280, Satt686, and Satt529 and that respectively mapped to the positions of 38.70 cM, 40.50 cM, and 41.29 cM on the map of Song et al., Theor. Appl. Genet. 109:122-128 (2004).

US Patent Application Publication 2009/0049565 discloses aphid resistance loci present in soybean Plant Introduction (PI) 1594427C that were associated with markers that mapped to linkage group J.

There is a need in the art of plant breeding to identify additional markers linked to quantitative trait loci associated with aphid resistance in soybean. There is in particular a need for numerous markers that are closely associated with aphid resistance QTLs in soybean that permit introgression of the aphid resistance QTL in the absence of extraneous linked DNA from the source germplasm containing the QTL. Additionally, there is a need for rapid, cost-efficient method to assay the absence or presence of aphid resistance loci in soybean.

### SUMMARY OF INVENTION

In certain embodiments, the present invention provides methods for producing aphid resistance in soybean plants, aphid resistant soybean plants, and polymorphic nucleic acids useful for identifying or producing aphid resistant soybean plants. In certain embodiments, the present invention further relates to methods to determine the presence or absence of quantitative trait loci conferring aphid resistance in soybean plants, including but not limited to exotic germplasm, populations, lines, elite lines, cultivars and varieties. In certain embodiments, the invention relates to methods that provide for identification of molecular markers associated with aphid resistance quantitative trait loci (QTL). In certain embodiments, the present invention relates to the use of molecular markers to screen and select for aphid resistance within soybean plants, including but not limited to exotic germplasm, populations, lines, elite lines, and varieties.

Methods of identifying a soybean plant that comprises a genotype associated with an aphid resistance phenotype are provided. In certain embodiments, these methods of identifying a soybean plant that comprises a genotype associated with an aphid resistance phenotype can comprise: i) detecting in the soybean plant an allele in at least one aphid resistance marker locus associated with the aphid resistance phenotype wherein the aphid resistance marker locus is in a linkage group J genomic region flanked by: a) loci NGMAX007665986 and NGMAX007668908; b) loci NGMAX007666844 and loci NGMAX007668908; c) loci NGMAX008369613 and loci NGMAX007668908; d) loci NGMAX007667203 and loci NGMAX007668908; e) loci NGMAX007667293 and NGMAX007668908; f) loci NGMAX007667294 and NGMAX007668908; g) loci NGMAX008369613 and NGMAX007668495; h) loci NGMAX007667203 and NGMAX007668495; i) loci NGMAX007667293 and NGMAX007668495; j) loci NGMAX007667294 and NGMAX007668495; k) loci NGMAX008369613 and NGMAX007668492; l) loci NGMAX007667203 and NGMAX007668492; m) loci NGMAX007667293 and NGMAX007668492; or, n) loci NGMAX007667294 and NGMAX007668492; and, ii) denoting that the plant comprises a genotype associated with an aphid resistance phenotype. In certain embodiments, these methods can further comprise the step of selecting the denoted plant from a population of plants. In certain embodiments of any of the aforementioned methods, a denoted plant does not comprise an allele of a Satt686, Satt280, Satt529, NS0115450, NS0122151, NS0125096, or NS0120948 marker that is associated with an aphid resistance phenotype. In certain embodiments of any of the aforementioned methods, a denoted plant does not comprise alleles of the Satt686, Satt280, Satt529, NS0115450, NS0122151, NS0125096, and NS0120948 markers that are associated with an aphid resistance phenotype. In certain embodiments of these methods, a denoted and/or selected plant exhibits an aphid resistance phenotype. In certain embodiments of any of the aforementioned methods, a genotype associated with an aphid resistance phenotype comprises at least one polymorphic allele of marker NS0202737 (SEQ ID NO: 35). In certain embodiments of any of the aforementioned methods, a genotype associated with an aphid resistance phenotype comprises at least one polymorphic allele of at least one marker selected from the group consisting of NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35).

Also provided are methods for obtaining a soybean plant comprising in its genome at least one aphid resistance locus. In certain embodiments, the methods for obtaining a soybean plant comprising in its genome at least one aphid resistance locus can comprise genotyping a plurality of soybean plants with respect to at least one aphid resistance locus in a linkage group J genomic region flanked by: a) loci NGMAX007665986 and NGMAX007668908; b) loci NGMAX007666844 and loci NGMAX007668908; c) loci NGMAX008369613 and loci NGMAX007668908; d) loci NGMAX007667203 and loci NGMAX007668908; e) loci NGMAX007667293 and NGMAX007668908; f) loci NGMAX007667294 and NGMAX007668908; g) loci NGMAX008369613 and NGMAX007668495; h) loci NGMAX007667203 and NGMAX007668495; i) loci NGMAX007667293 and NGMAX007668495; j) loci NGMAX007667294 and NGMAX007668495; k) loci NGMAX008369613 and NGMAX007668492; l) loci NGMAX007667203 and NGMAX007668492; m) loci NGMAX007667293 and NGMAX007668492; or, n) loci NGMAX007667294 and NGMAX007668492; and selecting a soybean plant comprising in its genome at least one aphid resistance locus comprising a genotype associated with an aphid resistance phenotype. In certain embodiments of these methods, the selected soybean plant exhibits aphid resistance. In certain embodiments of any of the aforementioned methods, the selected soybean plant does not comprise an allele of a Satt686, Satt280, Satt529, NS0115450, NS0122151, NS0125096, or NS0120948 marker that is associated with an aphid resistance phenotype. In certain embodiments of any of the aforementioned methods, the selected soybean plant does not comprise alleles of the Satt686, Satt280, Satt529, NS0115450, NS0122151, NS0125096, and NS0120948 markers that are associated with an aphid resistance phenotype. In certain embodiments of any of the aforementioned methods, the methods can further comprise the step of assaying for the presence of at least one additional marker, wherein the additional marker is either linked or unlinked to a linkage group J genomic region flanked by any one of the loci sets of (a), (b), (c), (d), (e), (f), (h), (i), (j), (k), (l), (m), or (n). In certain embodiments of the aforementioned methods, the methods can further comprise assaying the selected plant of step (ii) for an aphid resistance phenotype. In certain embodiments of any of the aforementioned methods, the methods can further comprise a step wherein an aphid resistance locus is genotyped for at least one polymorphic allele of marker NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35). In certain embodiments of any of the aforementioned methods, the methods can further comprise a step wherein an aphid resistance locus is genotyped for at least one polymorphic allele of at least one marker selected from the group consisting of NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35). In certain embodiments of any of the aforementioned methods, the at least one polymorphic allele is selected from the group consisting of a GG allele of NGMAX007666919 (SEQ ID NO: 16), an AA allele of NGMAX007666921 (SEQ ID NO: 17), a GG allele of NGMAX008369613 (SEQ ID NO: 23), a GG allele of NGMAX008369615 (SEQ ID NO: 28), a TT allele of NGMAX007667202 (SEQ ID: 29), a GG allele of NGMAX008383011 (SEQ ID: 34), and a CC allele of NS0202737 (SEQ ID NO: 35).

Also provided are methods for identifying a soybean plant comprising in its genome at least one introgressed aphid resistance locus. In certain embodiments, methods for identifying a soybean plant comprising in its genome at least one introgressed aphid resistance locus can comprise crossing a first soybean plant with a second soybean plant comprising: i) an aphid resistance locus in a linkage group J genomic region flanked by: a) loci NGMAX007665986 and NGMAX007668908; b) loci NGMAX007666844 and loci NGMAX007668908; c) loci NGMAX008369613 and loci NGMAX007668908; d) loci NGMAX007667203 and loci NGMAX007668908; e) loci NGMAX007667293 and NGMAX007668908; f) loci NGMAX007667294 and NGMAX007668908; g) loci NGMAX008369613 and NGMAX007668495; h) loci NGMAX007667203 and NGMAX007668495; i) loci NGMAX007667293 and NGMAX007668495; j) loci NGMAX007667294 and NGMAX007668495; k) loci NGMAX008369613 and NGMAX007668492; l) loci NGMAX007667203 and NGMAX007668492; m) loci NGMAX007667293 and NGMAX007668492; or, n) loci NGMAX007667294 and NGMAX007668492; and at least one additional polymorphic locus located outside of the linkage group J region, to obtain a population of soybean plants segregating for the aphid resistance loci and the at least one additional polymorphic locus; and ii) detecting the polymorphic nucleic acid in at least one soybean plant from the population of soybean plants, wherein the one soybean plant lacks the additional polymorphic locus, thereby identifying a soybean plant comprising in its genome at least one introgressed aphid resistance locus. In certain embodiments, these methods can further comprise the step of selecting the one soybean plant, thereby obtaining a soybean plant comprising in its genome at least one introgressed aphid resistance locus. In certain embodiments of any of the aforementioned methods, the identified or selected soybean plant does not comprise an allele of a Satt686, Satt280, Satt529, NS0115450, NS0122151, NS0125096, or NS0120948 marker that is associated with an aphid resistance phenotype. In certain embodiments of any of the aforementioned methods, an identified or selected soybean plant does not comprise alleles of the Satt686, Satt280, Satt529, NS0115450 NS0122151, NS0125096, and NS0120948 markers that are associated with an aphid resistance phenotype. In certain embodiments of any of the aforementioned methods, the aphid resistance locus comprises at least one polymorphic allele of at least one marker in a genomic region of the linkage group J region that is flanked by loci NGMAX007667294 and NGMAX007668492. In certain embodiments of any of the aforementioned methods, the polymorphic nucleic acid detected in step (ii) is detected with marker NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35). In certain embodiments of any of the aforementioned methods, the polymorphic nucleic acid is detected with at least one marker selected from the group consisting of NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35). In certain embodiments of any of the aforementioned methods, the polymorphic nucleic acid is detected is selected from the group consisting of a GG allele of NGMAX007666919 (SEQ ID NO: 16), an AA allele of NGMAX007666921 (SEQ ID NO: 17), a GG allele of NGMAX008369613 (SEQ ID NO: 23), a GG allele of NGMAX008369615 (SEQ ID NO: 28), a TT allele of NGMAX007667202 (SEQ ID: 29), a GG allele of NGMAX008383011 (SEQ ID: 34), and a CC allele of NS0202737 (SEQ ID NO: 35). In certain embodiments of any of the aforementioned methods, the identified or the selected plant is aphid resistant. In certain embodiments of any of the aforementioned methods, the identified or selected plant is assayed for aphid resistance. In certain embodiments of any of the aforementioned methods, the additional polymorphic locus is detected with a genotypic marker, a phenotypic marker, or both. In certain embodiments of any of the aforementioned methods, the additional polymorphic locus is a linked polymorphic locus located on linkage group J but not within the linkage group J genomic region flanked by any one of markers (a)-(m), or (n). In certain embodiments of any of the aforementioned methods, the linked polymorphic locus is detected with at least one marker that is located within a genomic region of the soybean genome flanked by: a) NGMAX007664762 and NGMAX007665668; and/or, b) NGMAX007669116 and Satt529. In certain embodiments of the aforementioned methods, wherein the linked polymorphic locus is detected with at least one marker selected from the group consisting of NGMAX007665590, NGMAX007665668, NGMAX007666264, NGMAX007666309, NGMAX007666777, NGMAX007666843, NGMAX007666869, NGMAX007666919, NGMAX007666921, NGMAX007666976, NGMAX007666977, NGMAX007667014, NGMAX007667071, NGMAX007667072, NGMAX007667077, NGMAX007667093, NGMAX007667095, NGMAX008369615, NGMAX007667202, and NGMAX007668494 and/or with at least one marker selected from the group consisting of NGMAX007669116, NGMAX007668903, and NGMAX007668494. Also provided herein are soybean plants obtainable by any of the aforementioned methods.

Soybean plants comprising plants comprising linkage group J genomic regions associated with an aphid resistance phenotype wherein immediately adjacent genomic regions and/or one or more adjacent genomic regions characteristic of soybean germplasms that lack the genomic regions associated with an aphid resistance phenotype and/or that are distinct from the germplasm from which the genomic region is derived are also provided. In certain embodiments, a soybean plant comprising i) an aphid resistance locus in a linkage group J region that is flanked by: a) loci NGMAX007665986 and NGMAX007668908; b) loci NGMAX007666844 and loci NGMAX007668908; c) loci NGMAX008369613 and loci NGMAX007668908; d) loci NGMAX007667203 and loci NGMAX007668908; e) loci NGMAX007667293 and NGMAX007668908; f) loci NGMAX007667294 and NGMAX007668908; g) loci NGMAX008369613 and NGMAX007668495; h) loci NGMAX007667203 and NGMAX007668495; i) loci NGMAX007667293 and NGMAX007668495; j) loci NGMAX007667294 and NGMAX007668495; k) loci NGMAX008369613 and NGMAX007668492; l) loci NGMAX007667203 and NGMAX007668492; m) loci NGMAX007667293 and NGMAX007668492; or, n) NGMAX007667294 and NGMAX007668492; and, ii) one or more polymorphic loci comprising alleles or combinations of alleles that are not found in a aphid resistant soybean varieties harboring the aphid resistance locus, and that are linked to the aphid resistance locus, wherein the soybean plant is aphid resistant is provided. In certain embodiments, the soybean plant does not comprise an allele of a Satt686, Satt280, Satt529, NS0115450, NS0122151, NS0125096, or NS0120948 marker that is associated with an aphid resistance phenotype. In certain embodiments, the soybean plant does not comprise alleles of the Satt686, Satt280, Satt529, NS0115450, NS0122151, NS0125096, and NS0120948 markers that are associated with an aphid resistance phenotype. In certain embodiments, the aphid resistance locus comprises an introgressed region of the soybean genome that is flanked by loci NGMAX007667294 and NGMAX007668492. In any of the aforementioned embodiments, the soybean plant can comprise an allele of marker NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35) that is associated with aphid resistance. In certain embodiments, the soybean plant can comprise an allele of at least one marker selected from the group consisting of NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35) that is associated with aphid resistance. In certain embodiments, the allele that is associated with aphid resistance is selected from the group consisting of a GG allele of NGMAX007666919 (SEQ ID NO: 16), an AA allele of NGMAX007666921 (SEQ ID NO: 17), a GG allele of NGMAX008369613 (SEQ ID NO: 23), a GG allele of NGMAX008369615 (SEQ ID NO: 28), a TT allele of NGMAX007667202 (SEQ ID: 29), a GG allele of NGMAX008383011 (SEQ ID: 34), and a CC allele of NS0202737 (SEQ ID NO: 35). In any of the aforementioned embodiments, the linked polymorphic loci comprising alleles or combinations of alleles that are not found in a aphid resistant soybean varieties harboring the aphid resistance locus can comprise alleles of at least one marker selected from the group consisting of NGMAX007665590, NGMAX007665668, NGMAX007666264, NGMAX007666309, NGMAX007666777, NGMAX007666843, NGMAX007666869, NGMAX007666919, NGMAX007666921, NGMAX007666976, NGMAX007666977, NGMAX007667014, NGMAX007667071, NGMAX007667072, NGMAX007667077, NGMAX007667093, NGMAX007667095, NGMAX008369615, NGMAX007667202, and NGMAX007668494 and/or comprise alleles of at least one marker selected from the group consisting of NGMAX007669116, NGMAX007668903, and NGMAX007668494.

Also provided herewith are isolated nucleic acid molecules comprising a nucleic acid molecule selected from the group consisting of an allele of marker NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35) that is associated with aphid resistance or aphid sensitivity. In certain embodiments, the allele that is associated with aphid resistance is selected from the group consisting of a GG allele of NGMAX007666919 (SEQ ID NO: 16), an AA allele of NGMAX007666921 (SEQ ID NO: 17), a GG allele of NGMAX008369613 (SEQ ID NO: 23), a GG allele of NGMAX008369615 (SEQ ID NO: 28), a TT allele of NGMAX007667202 (SEQ ID: 29), a GG allele of NGMAX008383011 (SEQ ID: 34), and a CC allele of NS0202737 (SEQ ID NO: 35). In certain embodiments, the nucleic acid can further comprise a detectable moiety. In certain embodiments, the detectable moiety can be selected from the group consisting of a chromophore, a fluorophore, and a hapten.

Also, methods of producing a population of soybean plants with an aphid resistance phenotype are provided. In certain embodiments, these methods of producing a population of soybean plants comprising a genotype associated with an aphid resistance phenotype can comprise: providing a first population of soybean plants, detecting in the soybean plants of the first population an allele in at least one aphid resistance marker locus associated with the aphid resistance phenotype wherein the aphid resistance marker locus is in a linkage group J genomic region flanked by or including: a) loci NGMAX007665986 and NGMAX007668908; b) loci NGMAX007666844 and loci NGMAX007668908; c) loci NGMAX008369613 and loci NGMAX007668908; d) loci NGMAX007667203 and loci NGMAX007668908; e) loci NGMAX007667293 and NGMAX007668908; f) loci NGMAX007667294 and NGMAX007668908; g) loci NGMAX008369613 and NGMAX007668495; h) loci NGMAX007667203 and NGMAX007668495; i) loci NGMAX007667293 and NGMAX007668495; j) loci NGMAX007667294 and NGMAX007668495; k) loci NGMAX008369613 and NGMAX007668492; l) loci NGMAX007667203 and NGMAX007668492; m) loci NGMAX007667293 and NGMAX007668492; or, n) loci NGMAX007667294 and NGMAX007668492; selecting one or more soybean plants exhibiting an allele in the at least one aphid resistance locus from the first population of soybean plants; and producing offspring from the one or more selected soybean plants. In certain embodiments of any of the aforementioned methods, the polymorphic nucleic acid is detected with at least one marker selected from the group consisting of NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35). In certain embodiments of any of the aforementioned methods, the polymorphic nucleic acid is detected is selected from the group consisting of a GG allele of NGMAX007666919 (SEQ ID NO: 16), an AA allele of NGMAX007666921 (SEQ ID NO: 17), a GG allele of NGMAX008369613 (SEQ ID NO: 23), a GG allele of NGMAX008369615 (SEQ ID NO: 28), a TT allele of NGMAX007667202 (SEQ ID: 29), a GG allele of NGMAX008383011 (SEQ ID: 34), and a CC allele of NS0202737 (SEQ ID NO: 35).

Further areas of applicability will become apparent from the description provided herein. It should be understood that the description and specific examples are intended for purposes of illustration only and are not intended to limit the scope of the present disclosure.

### DESCRIPTION OF INVENTION

### I. Definitions

As used herein, an “allele” refers to one of two or more alternative forms of a genomic sequence at a given locus on a chromosome. When all the alleles present at a given locus on a chromosome are the same, that plant is homozygous at that locus. If the alleles present at a given locus on a chromosome differ, that plant is heterozygous at that locus.

As used herein, the term “aphid” refers to any of various small, soft-bodied, plant-sucking insects of the Order Homoptera, further of the family Aphididae, wherein examples of Aphididae include but are not limited to the genus of Acyrthosiphon, Allocotaphis, Amphorophora, Anoecia, Anuraphis, Aphidounguis, Aphidura, Aphis, Asiphonaphis, Astegopteryx, Aulacorthum, Betacallis, Betulaphis, Boernerina, Brachycaudus, Brachycorynella, Brevicoryne, Calaphis, Callipterinella, Callipterus, Cavariella, Cerataphis, Ceratovacuna, Chaetomyzus, Chaetosiphon, Chaitophorus, Chaitoregma, Chromaphis, Cinara, Clethrobius, Clydesmithia, Coloradoa, Cornaphis, Cryptomyzus, Crypturaphis, Doralis, Doraphis, Drepanaphis, Drepanosiphoniella, Drepanosiphum, Dysaphis, Eomacrosiphum, Epipemphigus, Ericolophium, Eriosoma, Essigella, Euceraphis, Eulachnus, Eumyzus, Eutrichosiphum, Fimbriaphis, Fullawaya, Geopemphigus, Glyphina, Gootiella, Greenidea, Grylloprociphilus, Hamamelistes, Hannabura, Hormaphis, Hyadaphis, Hyalomyzus, Hyalopterus, Hyperomyza, Hyperomyzus, Hysteroneura, Illinola, Indiaphis, Indomasonaphis, Kakimia, Lachnus, Laingia, Lambersaphis, Latgerina, Longicaudus, Longistigma, Macromyzus, Macrosiphoniella, etc. while even further any one or more of the following genus species of Aphididae, examples of which including soybean aphid Aphis glycines, Bean aphid Aphis fabae, Cotton aphid Aphis gossypii, Rose aphid Macrosiphun rosae, green peach aphid Myzus persicae, corn leaf aphid Rhopalosiphum maidis, spotted alfalfa aphid Therioaphis maculata, wooly apple aphid Eriosoma lanigerum and the like.

As used herein, the term “antixenosis” refers to the ability of a plant to ability to repel insects, causing a reduction in egg laying and feeding.

As used herein, the term “antibiosis” refers the ability of a plant to reduce survival, growth, or reproduction of insects that feed on it.

As used herein, the term “bulk” refers to a method of managing a segregating population during inbreeding that involves growing the population in a bulk plot, harvesting the self pollinated seed of plants in bulk, and using a sample of the bulk to plant the next generation.

As used herein, the term “comprising” means “including but not limited to”.

As used herein, the term “denoting” when used in reference to a plant genotype refers to any method whereby a plant is indicated to have a certain genotype. Such indications of a certain genotype include, but are not limited to, any method where a plant is physically marked or tagged. Physical markings or tags that can be used include, but not limited to, a barcode, a radio-frequency identification (RFID), a label or the like. Indications of a certain genotype also include, but are not limited to, any entry into any type of written or electronic database whereby the plant's genotype is provided.

As used herein, the term “locus” refers to a position on a genomic sequence that is usually found by a point of reference; e.g., a short DNA sequence that is a gene, or part of a gene or intergenic region. A locus may refer to a nucleotide position at a reference point on a chromosome, such as a position from the end of the chromosome.

As used herein, “linkage group J” corresponds to the soybean linkage group J described in Choi, et al., Genetics. 2007 May; 176(1): 685-696. Linkage group J, as used herein, also corresponds to soybean chromosome 16 (as described on the World Wide Web at soybase.org/LG2Xsome.php).

As used herein, “polymorphism” means the presence of one or more variations of a nucleic acid sequence at one or more loci in a population of at least two members. The variation can comprise but is not limited to one or more nucleotide base substitutions, the insertion of one or more nucleotides, a nucleotide sequence inversion, and/or the deletion of one or more nucleotides.

As used herein, “genotype” means the genetic component of the phenotype and it can be indirectly characterized using markers or directly characterized by nucleic acid sequencing.

As used herein, the term “introgressed”, when used in reference to a genetic locus, refers to a genetic locus that has been introduced into a new genetic background. Introgression of a genetic locus can thus be achieved through both plant breeding methods or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion. In certain embodiments, introgression could thus be achieved by substitution of an aphid susceptibility locus with a corresponding aphid resistance locus or by conversion of a locus from a aphid susceptible genotype to a aphid resistance genotype.

As used herein, “linkage” refers to relative frequency at which types of gametes are produced in a cross. For example, if locus A has genes “A” or “a” and locus B has genes “B” or “b” and a cross between parent I with AABB and parent B with aabb will produce four possible gametes where the genes are segregated into AB, Ab, aB and ab. The null expectation is that there will be independent equal segregation into each of the four possible genotypes, i.e. with no linkage ¼ of the gametes will of each genotype. Segregation of gametes into a genotypes differing from ¼ are attributed to linkage.

As used herein, the termed “linked”, when used in the context of markers and/or genomic regions, means that the markers and/or genomic regions are located on the same linkage group or chromosome.

As used herein, “marker” means a detectable characteristic that can be used to discriminate between organisms. Examples of such characteristics include, but are not limited to, genetic markers, biochemical markers, fermentation yield, fermentation efficiency, energy yield, secondary compounds, metabolites, morphological characteristics, and agronomic characteristics.

As used herein, “marker assay” means a method for detecting a polymorphism at a particular locus using a particular method. Marker assays thus include, but are not limited to, measurement of at least one phenotype (such as seed color, flower color, or other visually detectable trait as well as any biochemical trait), restriction fragment length polymorphism (RFLP), single base extension, electrophoresis, sequence alignment, allelic specific oligonucleotide hybridization (ASO), random amplified polymorphic DNA (RAPD), microarray-based polymorphism detection technologies, and the like.

As used herein, “phenotype” means the detectable characteristics of a cell or organism which can be influenced by gene expression.

As used herein, the phrase “isolated nucleic acid molecule”, be it a naturally occurring molecule or otherwise, refers to a nucleic acid molecule where the covalent bonds between that nucleic acid and other native nucleic acids that adjoin the isolated nucleic acid in its naturally occurring state have been broken or have been replaced with covalent bonds to non-native nucleic acids. An isolated nucleic acid molecule can be the predominant species present in a preparation. In certain embodiments, an isolated nucleic acid molecule can also be at least about 60% free, at least about 75% free, at least about 90% free, and at least about 95% free from other molecules (exclusive of solvent). The phrase “isolated nucleic acid molecule” thus does not encompass nucleic acid molecules present in their native chromosomal locations.

As used herein, “quantitative trait locus (QTL)” means a locus that controls to some degree numerically representable traits that are usually continuously distributed.

As used herein, the term “soybean” refers to Glycine max and includes all plant varieties that can be bred with soybean, including wild soybean species. In certain embodiments, soybean plants from the species Glycine max and the subspecies Glycine max L. ssp. max or Glycine max ssp. formosana can be genotyped using the compositions and methods of the present invention. In an additional aspect, the soybean plant is from the species Glycine soja, otherwise known as wild soybean, can be genotyped using these compositions and methods. Alternatively, soybean germplasm derived from any of Glycine max, Glycine max L. ssp. max, Glycine max ssp. Formosana, and/or Glycine soja can be genotyped using compositions and methods provided herein.

As used herein, the term “single nucleotide polymorphism,” also referred to by the abbreviation “SNP,” means a polymorphism at a single site wherein the polymorphism constitutes any or all of a single base pair change, an insertion of one or more base pairs, and/or a deletion of one or more base pairs.

As used herein, the phrase “soybean aphid” refers to any aphid that is found on and feeds on a soybean plant. Aphids that feed on soybean include, but are not limited to, Aphis glycines, Aphis glycines Matasamura, and the bean aphid Aphis fabae.

As used herein, the phrase “soybean aphid resistance” refers to any form of resistance to an aphid that is found on and feeds on a soybean plant. Soybean aphid resistance thus includes, but is not limited to, antibiosis, antixenosis, tolerance, or any combination thereof.

As used herein, the term “tolerance”, when used in the context of aphid resistance, refers to the ability of a soybean plant to exhibit a reduction in deleterious effects caused by aphid feeding.

### II. Description: Overview

In accordance with the present invention, Applicants have discovered genomic regions, associated markers, and associated methods for identifying and associating genotypes that affect an aphid resistance trait. For example, in one embodiment, a method of the invention comprises screening for genotypes associated with aphid resistance within soybean plants, including but not limited to exotic germplasm, populations, lines, elite lines, and varieties, and identifying or selecting for plants comprising the genotypes associated with aphid resistance.

The use of markers to infer a phenotype of interest results in the economization of a breeding program by substituting costly, time-intensive phenotyping assays with genotyping assays. Further, breeding programs can be designed to explicitly drive the frequency of specific, favorable phenotypes by targeting particular genotypes (U.S. Pat. No. 6,399,855). Fidelity of these associations may be monitored continuously to ensure maintained predictive ability and, thus, informed breeding decisions (US Patent Application 2005/0015827). In this case, costly, time-intensive phenotyping assays required for determining if a plant or plants contains a genomic region associated with an aphid resistance phenotype can be supplanted by genotypic assays that provide for identification of a plant or plants that contain the desired genomic region.

### III. A Genomic Region Associated with an Aphid Resistance Phenotype

Provided herewith is a soybean genomic region that is shown herein to be associated with a desirable aphid resistance phenotype when present in certain allelic forms.

A soybean genomic region provided that can be associated with a desirable aphid resistance phenotype when present in certain allelic forms is located on the telomere proximal end of the short arm of soybean linkage group J (chromosome 16). A series of markers useful in practicing the methods of this invention are provided herewith in Table 1. Additional markers useful in the practice of the invention are provided herewith in Table 2. Table 2 provides the Table 1 markers, additional nucleic acid markers or loci that have been disclosed in various databases, the relative positions of the markers on a physical map of linkage group J (Glycine max chromosome 16), and sources for the markers.

TABLE 1
Markers spanning a genomic region associated with
a desirable aphid resistance phenotype

| | | | Allelic form(s) |
|---|---|---|---|
| | | | Associated |
| | | | with Aphid |
| | | | Resistance |
| Marker or Locus Name | SEQ ID NO: | Map Position¹ | Phenotype² |
| NS0125096³ | 6 | 47.3 | TT |
| NGMAX007665590 | 7 | 47.5 | TT⁴ |
| NGMAX007665986 | 9 | 48.1 | CC⁴ |
| NGMAX007666844 | 14 | 49.4 | TT⁴ |
| NGMAX007666919 | 16 | 49.6 | GG⁴ |
| NGMAX007666921 | 17 | 49.6 | AA⁴ |
| NGMAX007667014 | 20 | 49.9 | AA⁴ |
| NGMAX008369613 | 23 | 49.9 | GG⁴ |
| NGMAX007667203 | 27 | 50.3 | CC⁴ |
| NGMAX008369615 | 28 | 50.3 | GG⁴ |
| NGMAX007667202 | 29 | 50.3 | TT⁴ |
| NGMAX007667293 | 30 | 50.5 | CC⁴ |
| NGMAX008383011 | 34 | 51.6 | GG⁴ |
| NS0202737 | 35 | 52.6 | CC⁴ |
| NGMAX007668492 | 36 | 52.9 | AA⁴ |
| NGMAX007668495 | 38 | 52.9 | GG⁴ |
| NGMAX007668908 | 40 | 53.4 | TT⁴ |
| NS0122151 | 42 | 53.8 | AA⁴ |
| Satt686 | 54 | 59.7 | |
| NS0115450 | 55 | 61 | TT⁴ |
| NS0120948 | 56 | 62.8 | AA⁴ |
| Satt280 | 60 | 73 | |
| Satt529 | 62 | 77.4 | |

¹The relative positions of the middle position of the listed markers or loci based on nucleotide positions on a physical map of soybean linkage group J (chromosome 16) of Table 2 (Appendix to the Specification) are provided where nucleotide position 0 (zero) is telomere proximal and nucleotide position 23096098 is centromere proximal. Polymorphic nucleotide bases are designated in the sequence listing provided herewith according to the WIPO Standard ST.25 (1998), as follows: r = g or a (purine); y = t/u or c (pyrimidine); m = a or c; (amino); k = g or t/u (keto); s = g or c (strong interactions 3 H-bonds); w = a or t/u (weak interactions 2H-bonds); b = g or c or t/u (not a); d = a or g or t/u (not c); h = a or c or t/u (not g); v = a or g or c (not t, not u); and n = a or g or c or t/u (unknown, or other; any.)
²Both the maternal and paternal alleles of the single nucleotide polymorphisms that can be associated with an aphid resistance phenotype are shown.
³Name of marker or locus. Satt is a satellite marker.
⁴The identified polymorphic allele of marker NS0125096 is located at nucleotide 140 of SEQ ID NO: 6
⁴The identified polymorphic allele of marker NS0202737 is located at nucleotide 347 of SEQ ID NO: 35
⁴The identified polymorphic allele of marker NS0122151 is located at nucleotide 63 of SEQ ID NO: 42
⁴The identified polymorphic allele of marker NS0115450 is located at nucleotide 416 of SEQ ID NO: 55
⁴The identified polymorphic allele of marker NS0120948 is located at nucleotide 110 of SEQ ID NO: 56
⁴The identified polymorphic allele of all NGMAX markers NGMAX007665590, NGMAX007665986, NGMAX007666844, NGMAX007666919, NGMAX007666921, NGMAX007667014, NGMAX008369613, NGMAX007667203, NGMAX008369615, NGMAX007667202, NGMAX007667293, NGMAX008383011, NGMAX007668492, NGMAX007668495, NGMAX007668908 is located at nucleotide 101 of the respective SEQ ID NO in Table 2 and Table 7.

Also provided herein are sub-regions of the linkage group J region that is flanked by loci NGMAX007665986 and NGMAX007668908) that are associated with an aphid resistance phenotype. Sub-regions of the linkage group J region associated with an aphid resistance phenotype include, but are not limited to sub-regions defined by any of the following sets of loci:
a) loci NGMAX007665986 and NGMAX007668908;
b) loci NGMAX007666844 and loci NGMAX007668908;
c) loci NGMAX008369613 and loci NGMAX007668908;
d) loci NGMAX007667203 and loci NGMAX007668908;
e) loci NGMAX007667293 and NGMAX007668908;
f) loci NGMAX007667294 and NGMAX007668908;
g) loci NGMAX008369613 and NGMAX007668495;
h) loci NGMAX007667203 and NGMAX007668495;
i) loci NGMAX007667293 and NGMAX007668495;
j) loci NGMAX007667294 and NGMAX007668495;
k) loci NGMAX008369613 and NGMAX007668492;
l) loci NGMAX007667203 and NGMAX007668492;
m) loci NGMAX007667293 and NGMAX007668492; or,
n) loci NGMAX007667294 and NGMAX007668492.

These loci flank a sub-region that spans telomere proximal nucleotide 5461684 to centromere proximal nucleotide 6144533 in the physical map of linkage group J provided in the Table 2 appendix to the specification. Polymorphisms located in this first sub-region that are associated with an aphid resistance phenotype can be detected with markers that include, but are not limited to marker NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35). Significantly, Table 1 shows that the linkage group J regions comprising aphid resistance loci that are provided herein are centromere proximal to and distinct from the linkage group J regions comprising aphid resistant loci that have been previously identified in U.S. Pat. No. 7,781,648. More specifically, the aphid resistance loci and associated markers for the identification thereof provided in the instant application are located between positions at about 48.1 to about 53.4 cM on the map of Table 1 whereas the aphid resistance loci and markers of U.S. Pat. No. 7,781,648 (Satt280, Satt686, and Satt529) map to centromere proximal regions between 59.7 to about 77.4 cM on the map of Table 1. Furthermore, Table 1 also shows that the aphid resistance loci and associated markers for the identification thereof provided in the instant application that are located between positions at about 48.1 to about 53.4 cM on the map of Table 1 do not encompass the linkage group J NS0125096, NS0122151, NS0115450, or NS0120948 markers disclosed in US Patent Application Publication 2009/0049565.

Additional genetic markers can be used either in conjunction with the markers provided in Table 1 and/or Table 2 or independently of the markers provided in Table 1 and/or Table 2 to practice the methods of the instant invention. Publicly available marker databases from which useful markers can be obtained include, but are not limited to, the soybase.org website on the internet (World Wide Web) that is administered by the United States Agricultural Research Service, the United States Department of Agriculture, and Iowa State University. Additional soybean markers that can be used and that have been described in the literature include, but are not limited to, Hyten et al., BMC Genomics. 11:38, 2010; Choi et al., Genetics. 176(1):685-96, 2007; Yoon et al., Theor Appl Genet. 2007 March; 114(5):885-99; and Hyten et al. Crop Sci. 2010 50: 960-968. Given the provision herein of a genomic region on linkage group J (chromosome 16) delimited or flanked by the telomere proximal loci NGMAX007665986, NGMAX00766684, NGMAX008369613, NGMAX007667203, NGMAX007667293, or NGMAX007667294, of Table 2 and the centromere proximal loci NGMAX007668908, NGMAX007668495, and NGMAX007668492 of Table 2 as well as an assortment of soybean germplasms exhibiting either an aphid susceptible or an aphid resistance phenotype, additional markers located either within or near this genomic region that are associated with these phenotypes can be obtained by merely typing the new markers in the various germplasms provided herewith. The genomic region on linkage group J (chromosome 16) delimited or flanked by the telomere proximal loci NGMAX007665986, NGMAX00766684, NGMAX008369613, NGMAX007667203, NGMAX007667293, or NGMAX007667294, of Table 2 and the centromere proximal loci NGMAX007668908, NGMAX007668495, and NGMAX007668492 of Table 2 can also be mapped relative to markers provided in any publicly available or other soybean physical or genetic map to place this genetic locus on that map.

TABLE 2
Additional Markers in Linkage Group J

| | | SEQ ID | | Middle | | |
|---|---|---|---|---|---|---|
| Marker Annotation | LG | NO | cM | Position | Start | Stop |
| NGMAX007664762 | J | 1 | 41 | 4829863 | 4829763 | 4829963 |
| NGMAX007664838 | J | 2 | 41.2 | 4848089 | 4847989 | 4848189 |
| NGMAX007664836 | J | 3 | 41.2 | 4848118 | 4848018 | 4848218 |
| NGMAX007665386 | J | 4 | 47 | 5345749 | 5345649 | 5345849 |
| NGMAX007665387 | J | 5 | 47 | 5345760 | 5345660 | 5345860 |
| NS0125096 | J | 6 | 47.3 | 4939038 | 4939662 | 4938414 |
| NGMAX007665590 | J | 7 | 47.5 | 5382624 | 5382524 | 5382724 |
| NGMAX007665668 | J | 8 | 47.5 | 5391706 | 5391606 | 5391806 |
| NGMAX007665986 | J | 9 | 48.1 | 5461784 | 5461684 | 5461884 |
| NGMAX007666264 | J | 10 | 48.8 | 5498204 | 5498104 | 5498304 |
| NGMAX007666309 | J | 11 | 48.9 | 5507390 | 5507290 | 5507490 |
| NGMAX007666777 | J | 12 | 49.4 | 5589901 | 5589801 | 5590001 |
| NGMAX007666843 | J | 13 | 49.4 | 5598753 | 5598653 | 5598853 |
| NGMAX007666844 | J | 14 | 49.4 | 5598913 | 5598813 | 5599013 |
| NGMAX007666869 | J | 15 | 49.6 | 5608083 | 5607983 | 5608183 |
| NGMAX007666919 | J | 16 | 49.6 | 5617834 | 5617734 | 5617934 |
| NGMAX007666921 | J | 17 | 49.6 | 5617848 | 5617748 | 5617948 |
| NGMAX007666976 | J | 18 | 49.8 | 5636018 | 5635918 | 5636118 |
| NGMAX007666977 | J | 19 | 49.8 | 5636183 | 5636083 | 5636283 |
| NGMAX007667014 | J | 20 | 49.9 | 5645083 | 5644983 | 5645183 |
| NGMAX007667071 | J | 21 | 49.9 | 5654262 | 5654162 | 5654362 |
| NGMAX007667072 | J | 22 | 49.9 | 5654344 | 5654244 | 5654444 |
| NGMAX008369613 | J | 23 | 49.9 | 5654461 | 5654361 | 5654561 |
| NGMAX007667077 | J | 24 | 49.9 | 5654468 | 5654368 | 5654568 |
| NGMAX007667093 | J | 25 | 49.9 | 5656336 | 5656236 | 5656436 |
| NGMAX007667095 | J | 26 | 49.9 | 5656452 | 5656352 | 5656552 |
| NGMAX007667203 | J | 27 | 50.3 | 5700021 | 5699921 | 5700121 |
| NGMAX008369615 | J | 28 | 50.3 | 5700008 | 5699908 | 5700108 |
| NGMAX007667202 | J | 29 | 50.3 | 5700111 | 5700011 | 5700211 |
| NGMAX007667293 | J | 30 | 50.5 | 5736555 | 5736455 | 5736655 |
| NGMAX007667292 | J | 31 | 50.5 | 5736617 | 5736517 | 5736717 |
| NGMAX007667295 | J | 32 | 50.5 | 5736709 | 5736609 | 5736809 |
| NGMAX007667294 | J | 33 | 50.5 | 5736717 | 5736617 | 5736817 |
| NGMAX008383011 | J | 34 | 51.6 | 5883668 | 5883568 | 5883768 |
| NS0202737 | J | 35 | 52.6 | 6025443 | 6020859 | 6030026 |
| NGMAX007668492 | J | 36 | 52.9 | 6087908 | 6087808 | 6088008 |
| NGMAX007668494 | J | 37 | 52.9 | 6088039 | 6087939 | 6088139 |
| NGMAX007668495 | J | 38 | 52.9 | 6088092 | 6087992 | 6088192 |
| NGMAX007668903 | J | 39 | 53.4 | 6144239 | 6144139 | 6144339 |
| NGMAX007668908 | J | 40 | 53.4 | 6144433 | 6144333 | 6144533 |
| NGMAX007669116 | J | 41 | 53.8 | 6199351 | 6199251 | 6199451 |
| NS0122151 | J | 42 | 53.8 | 5497446 | 5497148 | 5497744 |
| NGMAX008369616 | J | 43 | 54.3 | 6273337 | 6273237 | 6273437 |
| NGMAX008369614 | J | 44 | 54.8 | 6341835 | 6341735 | 6341935 |
| NGMAX007670330 | J | 45 | 55.4 | 6420377 | 6420277 | 6420477 |
| NGMAX008369617 | J | 46 | 55.5 | 6438676 | 6438576 | 6438776 |
| NGMAX008369618 | J | 47 | 55.5 | 6438695 | 6438595 | 6438795 |
| NGMAX007670824 | J | 48 | 55.8 | 6484596 | 6484496 | 6484696 |
| NGMAX007671248 | J | 49 | 55.9 | 6567207 | 6567107 | 6567307 |
| NGMAX007671280 | J | 50 | 55.9 | 6576371 | 6576271 | 6576471 |
| NGMAX007671348 | J | 51 | 55.9 | 6587505 | 6587405 | 6587605 |
| NGMAX007671426 | J | 52 | 55.9 | 6604800 | 6604700 | 6604900 |
| NGMAX007671425 | J | 53 | 55.9 | 6605096 | 6604996 | 6605196 |
| Satt686 | J | 54 | 59.7 | 13657187 | 13657046 | 13657328 |
| NS0115450 | J | 55 | 61 | 6683797 | 6683353 | 6684241 |
| NS0120948 | J | 56 | 62.8 | 8480218 | 8479913 | 8480523 |
| NS0119584 | J | 57 | 65.2 | 10907066 | 10907404 | 10906727 |
| NS0093252 | J | 58 | 72.8 | 18517177 | 18516957 | 18517397 |
| NS0093989 | J | 59 | 72.8 | 18517692 | 18517470 | 18517914 |
| Satt280 | J | 60 | 73 | 18769579 | 18769464 | 18769693 |
| NS0203255 | J | 61 | 76.3 | 22052294 | 22051991 | 22052596 |
| Satt529 | J | 62 | 77.4 | 23095994 | 23095889 | 23096098 |

Sequences for genes provided above can be obtained from either the listing of sequences provided herewith in the Summary Table of Nucleic Acid Sequences in the Examples (Table 8), or on the World Wide Web (or Internet) using the identifiers provided in Column 1 (Locus/Display Name) from the following internet locations:
a) “soybase.org” (described in Grant et al., Nucleic Acids Research, 2010, Vol. 38, Database issue D843-D846) or soybase.org/gbrowse/cgi-bin/gbrowse/gmax1.01/ (see Hyten D L, Choi I-Y, Song Q, Specht J E, Carter T E et al. (2010) A high density integrated genetic linkage map of soybean and the development of a 1,536 Universal Soy Linkage Panel for QTL mapping. Crop Science 50:960-968. (Crop Science); and Hyten D L, Cannon S B, Song Q, Weeks N, Fickus E W et al. (2010) High-throughput SNP discovery through deep resequencing of a reduced representation library to anchor and orient scaffolds in the soybean whole genome sequence. BMC Genomics 11(1): 38);
b) “phytozome.net” or “phytozome.net/cgi-bin/gbrowse/soybean/?name=Gm09”;
c) “www.plantgdb.org” or “plantgdb.org/GmGDB/ (Assembly version Glyrna1.170 (April 2009)”; and,
d) “ncbi.nlm.nih.gov/sites/entrez” and subsites “ncbi.nlm.nih.gov/nucest”, “ncbi.nlm.nih.gov/dbEST”, “ncbi.nlm.nih.gov/genbank/”, “.ncbi.nlm.nih.gov/sites/genome”, “ncbi.nlm.nih.gov/unigene”, and “ncbi.nlm.nih.gov/UniGene/UGOrg.cgi?TAXID=3847”.

### IV. Identification of Plants Exhibiting the Aphid Resistant Phenotype

To observe the presence or absence of the aphid resistance phenotypes, soybean plants comprising genotypes of interest can be exposed to aphids in seedling stages, early to mid-vegetative growth stages, or in early reproductive stages. The design and execution of aphid exposure experiments to assess antibiosis, antixenosis, and tolerance have been described in numerous publications including, but not limited to, Pierson et al. (J. Econ. Entomol. 103(4): 1405-1411 (2010); Diaz-Montano et al. J. Econ. Entomol. 99: 1884-1889 (2006). In general, antibiosis can be determined by measuring any aspect of aphid survival and/or fecundity following exposure to the plants. In certain embodiments, nymphs can be counted a suitable number of days past infestation. Antixenosis can be determined in “choice experiments” where the aphids are exposed to at least two plants, permitted to “choose” a plant for feeding, and the number of aphids per plant and/or aphid damage to the plants is scored. Tolerance can be determined by exposing the plants to aphids and measuring any plant growth feature that is impacted by aphid infestation. In certain embodiments, tolerance can be assessed by measuring a soybean yield parameter. Soybean yield parameters that can be examined to assess aphid tolerance include, but are not limited to, average seed weight, average seeds per pod, average number of pods per plant, chlorophyll content

A rating scale that evaluates the degree of aphid resistance can also be employed to identify “aphid susceptible” and “aphid resistance” plants. An exemplary and non-limiting scale for evaluating the aphid susceptibility phenotype is as follows, where the low numbers correspond to an “aphid resistance” phenotype and the high numbers correlate to an “aphid susceptible” phenotype.

An exemplary rating and damage system that can be used is a 1-4 visual rating scale as described in Table 3.

TABLE 3
Description of rating scale used for aphid resistance phenotyping

| Rating | Description | Symptoms |
|---|---|---|
| 0 | Very Resistant | No aphids |
| 1 | Resistant | Fewer than 100 aphids |
| 2 | Moderately Resistant | 101-300 aphids |
| 3 | Moderately Susceptible | 300-800 aphids |
| 4 | Susceptible | >800 aphids |

In certain embodiments, the plants can be assigned a damage index (DI), which is calculated using the following formula:

$$DI = \frac{\sum ( each scale \times no . of plants in the scale )}{4 \times total no . of plants evaluated} \times 100$$

In this formula, a higher damage index corresponds to a more susceptible plant.

In other embodiments, a 1-5 scale can be used. A 1-5 scale, where 1 is 10% yellowing discoloration, leaf distortion, plant stunting, and desiccation; 2 is 11-30% yellowing discoloration, leaf distortion, plant stunting, and desiccation; 3 is 31-50% yellowing discoloration, leaf distortion, plant stunting, and desiccation; 4 is 51-75% yellowing discoloration, leaf distortion, plant stunting, and desiccation; and 5 is 76% of leaf area with yellowing discoloration, leaf distortion, plant stunting, desiccation, or dead tissue is described by Pierson et al. (J. Econ. Entomol. 103(4): 1405-1411 (2010). Under this system, plants exposed to aphids are characterized as either: HS, highly susceptible (damage rating ≧4); MS, moderately susceptible (damage rating ≧3 but <4); MR, moderately resistant (damage rating ≧1 but <3); and HR, highly resistant (damage rating=1) (Pierson et al. (J. Econ. Entomol. 103(4): 1405-1411 (2010); Heng-Moss et al., J. Econ. Entomol. 95: 1054-1058 (2002)). Other rating scales that can be used to identify aphid resistant and susceptible plants are also described by Hill et al. Crop Set 44: 98-106 (2004).

### V. Introgression of a Genomic Region Associated with a Aphid Resistance Phenotype

Also provided herewith are unique soybean germplasms comprising an introgressed genomic region that is associated with an aphid resistance phenotype and methods of obtaining the same. Marker-assisted introgression involves the transfer of a chromosomal region, defined by one or more markers, from one germplasm to a second germplasm. Offspring of a cross that contain the introgressed genomic region can be identified by the combination of markers characteristic of the desired introgressed genomic region from a first germplasm (i.e. such as a aphid resistance germplasm) and both linked and unlinked markers characteristic of the desired genetic background of a second germplasm (i.e. an aphid susceptible germplasm). In addition to the markers provided herewith that identify alleles of genomic region that is associated with a aphid resistance phenotype, flanking markers that fall on both the telomere proximal end of the genomic region on linkage group J (chromosome 16) and the centromere proximal end of the linkage group J (chromosome 16) genomic region are also provided in Tables 1, 2, and 4. Such flanking markers are useful in a variety of breeding efforts that include, but are not limited to, introgression of the genomic region associated with a aphid resistance phenotype into a genetic background comprising markers associated with germplasm that ordinarily contains the allelic forms of the genomic region that is associated with a “aphid susceptible” phenotype. Numerous markers that are linked and either immediately adjacent or adjacent to a linkage group J aphid resistance QTL in soybean that permit introgression of the aphid resistance QTL in the absence of extraneous linked DNA from the source germplasm containing the QTL are provided herewith. In certain embodiments, the linked and immediately adjacent markers are within about 105 kilobases (kB), 80 kB, 60 kB, 50 kB, 40 kB, 30 kB, 20 kB, 10 kB, 5 kB, 1 kB, 0.5 kB, 0.2 kB, or 0.1 kB of the introgressed genomic region. In certain embodiments, the linked and adjacent markers are within 1,000 kB, 600 kB, 500 kB, 400 kB, 300 kB, 200 kB, or 150 kB of the introgressed genomic region. In certain embodiments, genomic regions comprising some or all of an aphid resistance QTL on linkage group J (chromosome 16) that are delimited by the following markers of Table 4 can be introgressed into the genomes of susceptible varieties by using markers that include, but are not limited to, adjacent markers and/or immediately adjacent markers provided in Tables 1, 2, or 4. Those skilled in the art will appreciate that when seeking to introgress a smaller genomic region comprising a aphid resistance locus of Table 4 that any of the telomere proximal or centromere proximal markers that are immediately adjacent to a larger genomic region comprising a aphid resistance locus can also be used to introgress that smaller genomic region.

TABLE 4
Genomic Regions containing Aphid Resistance Loci , Exemplary Adjacent
Markers, and Exemplary Immediately Adjacent Markers for Introgression

| Genomic Region Comprising | | |
|---|---|---|
| a linkage group J Aphid | Immediately Adjacent | Immediately Adjacent |
| Resistance Locus | Telomere Proximal Markers¹ | Centromere Proximal Markers² |
| a) NGMAX007665986 and | NGMAX007665590 | NGMAX007669116 |
| NGMAX007668908; | NGMAX007665668 | |
| b) loci | NGMAX007666264 | NGMAX007669116 |
| NGMAX007666844 | NGMAX007666309 | |
| and loci | NGMAX007666777 | |
| NGMAX007668908; | NGMAX007666843 | |
| c) loci | NGMAX007666869 | NGMAX007669116 |
| NGMAX008369613 | NGMAX007666919 | |
| and loci | NGMAX007666921 | |
| NGMAX007668908; | NGMAX007666976 | |
| | NGMAX007666977 | |
| | NGMAX007667014 | |
| | NGMAX007667071 | |
| | NGMAX007667072 | |
| d) loci | NGMAX007667077 | NGMAX007669116 |
| NGMAX007667203 | NGMAX007667093 | |
| and loci | NGMAX007667095 | |
| NGMAX007668908; | | |
| e) loci | NGMAX008369615 | NGMAX007669116 |
| NGMAX007667293 | NGMAX007667202 | |
| and | | |
| NGMAX007668908; | | |
| f) loci | NGMAX007667292 | NGMAX007669116 |
| NGMAX007667294 | NGMAX007667295 | |
| and | | |
| NGMAX007668908; | | |
| g) loci | NGMAX007666869 | NGMAX007668903 |
| NGMAX008369613 | NGMAX007666919 | |
| and | NGMAX007666921 | |
| NGMAX007668495; | NGMAX007666976 | |
| | NGMAX007666977 | |
| | NGMAX007667014 | |
| | NGMAX007667071 | |
| | NGMAX007667072 | |
| h) loci | NGMAX007667077 | NGMAX007668903 |
| NGMAX007667203 | NGMAX007667093 | |
| and | NGMAX007667095 | |
| NGMAX007668495; | | |
| i) loci | NGMAX008369615 | NGMAX007668903 |
| NGMAX007667293 | NGMAX007667202 | |
| and | | |
| NGMAX007668495; | | |
| j) loci | NGMAX007667292 | NGMAX007668903 |
| NGMAX007667294 | NGMAX007667295 | |
| and | | |
| NGMAX007668495; | | |
| k) loci | NGMAX007666869 | NGMAX007668494 |
| NGMAX008369613 | NGMAX007666919 | |
| and | NGMAX007666921 | |
| NGMAX007668492; | NGMAX007666976 | |
| | NGMAX007666977 | |
| | NGMAX007667014 | |
| | NGMAX007667071 | |
| | NGMAX007667072 | |
| l) loci | NGMAX007667077 | NGMAX007668494 |
| NGMAX007667203 | NGMAX007667093 | |
| and | NGMAX007667095 | |
| NGMAX007668492; | | |
| m) loci | NGMAX008369615 | NGMAX007668494 |
| NGMAX007667293 | NGMAX007667202 | |
| and | | |
| NGMAX007668492; | | |
| or, | | |
| n) loci | NGMAX007667292 | NGMAX007668494 |
| NGMAX007667294 | NGMAX007667295 | |
| and | | |
| NGMAX007668492 | | |

¹Closely associated markers located between the telomere and the genomic region containing an aphid resistance locus.
²Closely associated markers located between the centromere and the genomic region containing an aphid resistance locus.

Provided herein are methods of introgressing any of the genomic regions comprising a linkage group J aphid resistance locus of Table 4 into soybean germplasm that lacks such a linkage group J aphid resistance locus. In certain embodiments, the soybean germplasm that lacks such a genomic region comprising linkage group J aphid resistance locus is aphid susceptible or has less than optimal levels of aphid resistance. In certain embodiments, the methods of introgression provided herein can yield soybean plants comprising introgressed genomic regions comprising a linkage group J aphid resistance locus of Table 4 where the immediately adjacent genomic DNA and/or some or all of the adjacent genomic DNA between the introgressed genomic region and the telomere or centromere will comprise allelic forms of the markers of Tables, 1, 2 or 4 that are characteristic of the germ plasm into which the genomic region is introgressed and distinct from the germplasm from which the genomic region is derived. In certain embodiments, the soybean germplasm into which the genomic region is introgressed is germplasm that lacks such a linkage group J aphid resistance locus. In certain embodiments, the soybean germplasm into which the genomic region is introgressed is germplasm that lacks such a linkage group J aphid resistance locus and is either aphid susceptible or has less than optimal levels of aphid resistance. In certain embodiments, the germplasm from which the linkage group J aphid resistance locus comprises PI594427C germplasm or germplasm derived therefrom.

Also provided herein are soybean plants produced by the aforementioned methods of introgression. In certain embodiments, such soybean plants will comprising introgressed genomic regions comprising a linkage group J aphid resistance locus of Table 4 where the immediately adjacent genomic DNA and/or some or all of the adjacent genomic DNA between the introgressed genomic region and the telomere or centromere will comprise allelic forms of the markers of Tables 1, 2, or 4 that are characteristic of the germ plasm into which the genomic region is introgressed and distinct from the germplasm from which the genomic region is derived. In an exemplary embodiment where a genomic region flanked by markers NGMAX007667294 and NGMAX007668492 is introgressed, plants comprising that linkage group J genomic region containing an aphid resistance locus wherein one or more of the adjacent telomere proximal markers NGMAX007665590, NGMAX007665668, NGMAX007666264, NGMAX007666309, NGMAX007666777, NGMAX007666843, NGMAX007666869, NGMAX007666919, NGMAX007666921, NGMAX007666976, NGMAX007666977, NGMAX007667014, NGMAX007667071, NGMAX007667072, NGMAX007667077, NGMAX007667093, and NGMAX007667095, and the adjacent centromere proximal marker NGMAX007669116, can comprise allelic forms that are characteristic of the germ plasm into which the genomic region is introgressed and/or that are distinct from the germplasm from which the genomic region is derived. In another exemplary embodiment where a genomic region flanked by markers NGMAX007667294 and NGMAX007668492 is introgressed, plants comprising that linkage group J genomic region containing an aphid resistance locus wherein one or more of the adjacent telomere proximal markers NGMAX007665590, NGMAX007665668, NGMAX007666264, NGMAX007666309, NGMAX007666777, NGMAX007666843, NGMAX007666869, NGMAX007666919, NGMAX007666921, NGMAX007666976, NGMAX007666977, NGMAX007667014, NGMAX007667071, NGMAX007667072, NGMAX007667077, NGMAX007667093, NGMAX007667095, NGMAX008369615, and NGMAX007667202, and adjacent centromere proximal markers NGMAX007669116 and/or NGMAX007668903, can comprise allelic forms that are characteristic of the germ plasm into which the genomic region is introgressed and/or that are distinct from the germplasm from which the genomic region is derived. In another exemplary embodiment where a genomic region flanked by markers NGMAX007667294 and NGMAX007668492 is introgressed, plants comprising that linkage group J genomic region containing an aphid resistance locus wherein immediately adjacent telomere proximal markers NGMAX007667292 and NGMAX007667295, wherein one or more of the adjacent telomere proximal markers NGMAX007665590, NGMAX007665668, NGMAX007666264, NGMAX007666309, NGMAX007666777, NGMAX007666843, NGMAX007666869, NGMAX007666919, NGMAX007666921, NGMAX007666976, NGMAX007666977, NGMAX007667014, NGMAX007667071, NGMAX007667072, NGMAX007667077, NGMAX007667093, NGMAX007667095, NGMAX008369615, and NGMAX007667202, immediately adjacent centromere proximal marker NGMAX007668494, and wherein one or more of the adjacent centromere proximal markers NGMAX007669116 and NGMAX007668903, can comprise allelic forms that are characteristic of the germ plasm into which the genomic region is introgressed and/or that are distinct from the germplasm from which the genomic region is derived.

Additional markers located on linkage group J (chromosome 16) and other chromosomes useful for introgressing a linkage group J soybean aphid resistance QTL are disclosed in US Patent Publication 2009/0049565. Publicly available marker databases from which additional useful markers located on linkage group J (chromosome 16) and other chromosomes can be obtained include, but are not limited to, the soybase.org website on the internet that is administered by the United States Agricultural Research Service, the United States Department of Agriculture, and Iowa State University. Soybean plants or germplasm comprising an introgressed genomic region that is associated with a aphid resistance phenotype wherein at least 10%, 25%, 50%, 75%, 90%, or 99% of the remaining genomic sequences carry markers characteristic of soybean plants or germplasm that are otherwise or ordinarily comprise a genomic region associated with the aphid susceptible phenotype are thus provided. Furthermore soybean plants comprising an introgressed region where closely linked regions adjacent and/or immediately adjacent to the linkage group J regions provided herewith that comprise genomic sequences carrying markers characteristic of soybean plants or germplasm that are otherwise or ordinarily comprise a genomic region associated with the aphid susceptible phenotype are also provided.

Soybean Plants Comprising a Genomic Region Associated with a Aphid Resistance Phenotype

Also provided herein are soybean plants comprising linkage group J genomic regions associated with an aphid resistance phenotype wherein immediately adjacent genomic regions and/or one or more adjacent genomic regions characteristic of soybean germplasms that lack the genomic regions associated with an aphid resistance phenotype and/or that are distinct from the germplasm from which the genomic region is derived: In certain embodiments, such plants can be produced by the aforementioned methods of introgression. In certain embodiments, soybean plants comprising a linkage group J aphid resistance locus of Table 4 where the immediately adjacent genomic DNA and/or some or all of the adjacent genomic DNA between the introgressed genomic region and the telomere or centromere will comprise allelic forms of the markers of Tables 1, 2, or 4 that are characteristic of germplasms that lack the linkage group J genomic regions of Table 4 comprising an aphid resistance phenotype and/or that are distinct from the germplasm from which the genomic region is derived.

In certain embodiments, aphid resistant plants comprising a linkage group J genomic region flanked by markers NGMAX007667294 and NGMAX007668492 are provided wherein one or more of the adjacent telomere proximal markers NGMAX007665590, NGMAX007665668, NGMAX007666264, NGMAX007666309, NGMAX007666777, NGMAX007666843, NGMAX007666869, NGMAX007666919, NGMAX007666921, NGMAX007666976, NGMAX007666977, NGMAX007667014, NGMAX007667071, NGMAX007667072, NGMAX007667077, NGMAX007667093, and NGMAX007667095, and the adjacent centromere proximal marker NGMAX007669116 comprise allelic forms that are characteristic of germplasms that lack the linkage group J genomic regions of Table 4 comprising an aphid resistance phenotype and/or that are distinct from the germplasm from which the genomic region is derived. In another exemplary embodiment, aphid resistant plants comprising linkage group J genomic region flanked by markers NGMAX007667294 and NGMAX007668492 are provided wherein one or more of the adjacent telomere proximal markers NGMAX007665590, NGMAX007665668, NGMAX007666264, NGMAX007666309, NGMAX007666777, NGMAX007666843, NGMAX007666869, NGMAX007666919, NGMAX007666921, NGMAX007666976, NGMAX007666977, NGMAX007667014, NGMAX007667071, NGMAX007667072, NGMAX007667077, NGMAX007667093, NGMAX007667095, NGMAX008369615, and NGMAX007667202, and adjacent centromere proximal markers NGMAX007669116 and/or NGMAX007668903 comprise allelic forms that are characteristic of the germ plasm into which the genomic region is introgressed and/or that are distinct from the germplasm from which the genomic region is derived. In certain embodiments, aphid resistant plants comprising a linkage group J genomic region flanked by markers NGMAX007667294 and NGMAX007668492 are provided wherein immediately adjacent telomere proximal markers NGMAX007667292 and NGMAX007667295, wherein one or more of the adjacent telomere proximal markers NGMAX007665590, NGMAX007665668, NGMAX007666264, NGMAX007666309, NGMAX007666777, NGMAX007666843, NGMAX007666869, NGMAX007666919, NGMAX007666921, NGMAX007666976, NGMAX007666977, NGMAX007667014, NGMAX007667071, NGMAX007667072, NGMAX007667077, NGMAX007667093, NGMAX007667095, NGMAX008369615, and NGMAX007667202, immediately adjacent centromere proximal marker NGMAX007668494, and wherein one or more of the adjacent centromere proximal markers NGMAX007669116 and NGMAX007668903, can comprise allelic forms that are characteristic of germplasms that lack the linkage group J genomic regions of Table 4 comprising an aphid resistance phenotype and/or that are distinct from the germplasm from which the genomic region is derived.

As used herein, a maturity group refers to an industry division of groups of varieties based range in latitude which the plant is best adapted and most productive. Soybean varieties are classified into 13 recognized maturity groups with the designations ranging from maturity groups 000, 00, 0, and I through X, wherein 000 represents the earliest maturing variety and X represents the latest maturing variety. Soybean plants in maturity groups 000 to IV have indeterminate plant habit, while soybean plants in maturity groups V through X have determinate plant habit. Herein, determinate growth habit refers to a cease vegetative growth after the main stem terminates in a cluster of mature pods. Herein, indeterminate growth habit refers to the development of leaves and flowers simultaneously throughout a portion of their reproductive period, with one to three pods at the terminal apex. Early maturity varieties (000 to IV) are adapted to northern latitudes with longer day lengths with the maturity designation increasing in southern latitudes with shorter day lengths

Herein, relative maturity refers to a soybean plant maturity group subdividing a maturity group into tenths, for example III.5. Relative maturity provided a more exact maturity. The number following the decimal point refers to the relative earliness or lateness with a maturity group, examples of which including IV.2 is an early group IV variety and IV.9 is a late group IV.

It is further understood that a soybean plant of the present invention may exhibit the characteristics of any relative maturity group. In an aspect, the relative maturity group is selected from the group consisting of 000.1-000.9, 00.1-00.9, 0.1-0.9, I.1-I.9, II.1-II.9, III.1-III.9, IV.1-IV.9, V.1-V.9, VI.1-VI.9, VII.1-VII.9, VIII.1-VIII.9, IX.1-IX.9, and X.1-X.9. The pollen for selected soybean plant can be cryopreserved and used in crosses with soybean lines from other maturity groups to introgress an aphid resistance locus in a line that would not normally be available for crossing in nature. Pollen cryopreservation techniques are well known in the art (Tyagi and Hymowitz, Cryo letters 24: 119-124 (2003), Liang et al. Acta Botanica Sinica 35: 733-738 (1993).

### VI. Soybean Donor Plants Comprising Genomic Region Associated with the Aphid Resistance Phenotypes

An aphid resistant QTL allele or alleles can be introduced from any plant that contains that allele (donor) to any recipient soybean plant. In one aspect, the recipient soybean plant can contain additional aphid resistant loci. In another aspect, the recipient soybean plant can contain a transgene. In another aspect, while maintaining the introduced QTL, the genetic contribution of the plant providing the aphid resistant QTL can be reduced by back-crossing or other suitable approaches. In one aspect, the nuclear genetic material derived from the donor material in the soybean plant can be less than or about 50%, less than or about 25%, less than or about 13%, less than or about 5%, 3%, 2% or 1%, but that genetic material contains the aphid resistant locus or loci of interest.

Plants containing one or more aphid resistant loci described can be donor plants. Aphid plants containing resistant loci can be identified and/or selected by using a nucleic acid molecule capable of detecting a marker polymorphism associated with resistance. Soybean donor plants comprising a genomic region containing a linkage group J aphid resistance locus include, but are not limited to, soybean Plant Introduction (PI) 594427C and derivatives thereof. In certain embodiments, a donor plant can be a susceptible line. In certain embodiments, a donor plant can also be a recipient soybean plant.

In certain embodiments, the soybean plants provided herein or used in the methods provided herein can comprise a transgene that confers tolerance to glyphosate. Transgenes that can confer tolerance to glyphosate include, but are not limited to, transgenes that encode glyphosate tolerant Class I EPSPS (5-enolpyruvylshikimate-3-phosphate synthases) enzymes or glyphosate tolerant Class II EPSPS (5-enolpyruvylshikimate-3-phosphate synthases) enzymes. Useful glyphosate tolerant EPSPS enzymes provided herein are disclosed in U.S. Pat. No. 6,803,501, RE39,247, U.S. Pat. No. 6,225,114, U.S. Pat. No. 5,188,642, and U.S. Pat. No. 4,971,908. In certain embodiments, the glyphosate tolerant soybean plants can comprise a transgene encoding a glyphosate oxidoreductase or other enzyme which degrades glyphosate. Glyphosate oxidoreductase enzymes had been described in U.S. Pat. No. 5,776,760 and US Reissue patent RE38,825. In certain embodiments the soybean plant can comprise a transgene encoding a glyphosate N-acetyltransferase gene that confers tolerance to glyphosate. In certain embodiments, the soybean plant can comprise a glyphosate n-acetyltransferase encoding transgene such as those described in U.S. Pat. No. 7,666,644. In still other embodiments, soybean plants comprising combinations of transgenes that confer glyphosate tolerance are provided. Soybean plants comprising both a glyphosate resistant EPSPS and a glyphosate N-acetyltransferase are also provided herewith. In certain embodiments, it is contemplated that the soybean plants used herein can comprise one or more specific genomic insertion(s) of a glyphosate tolerant transgene including, but not limited to, as those found in: i) MON89788 soybean (deposited under ATCC accession number PTA-6708 and described in US Patent Application Publication Number 20100099859), ii) GTS 40-3-2 soybean (Padgette et al., Crop Sci. 35: 1451-1461, 1995), iii) event 3560.4.3.5 soybean (seed deposited under ATCC accession number PTA-8287 and described in US Patent Publication 20090036308), or any combination of i (MON89788 soybean), ii (GTS 40-3-2 soybean), and iii (event 3560.4.3.5 soybean).

An aphid resistance QTL of the present invention may also be introduced into an soybean line comprising one or more transgenes that confer tolerance to herbicides including, but not limited to, glufosinate, dicamba, chlorsulfuron, and the like, increased yield, insect control, fungal disease resistance, virus resistance, nematode resistance, bacterial disease resistance, mycoplasma disease resistance, modified oils production, high oil production, high protein production, germination and seedling growth control, enhanced animal and human nutrition, low raffinose, environmental stress resistant, increased digestibility, industrial enzymes, pharmaceutical proteins, peptides and small molecules, improved processing traits, improved flavor, nitrogen fixation, hybrid seed production, reduced allergenicity, biopolymers, and biofuels among others. These agronomic traits can be provided by the methods of plant biotechnology as transgenes in soybean.

In certain embodiments, it is contemplated that genotypic assays that provide for non-destructive identification of the plant or plants can be performed either in seed, the emergence stage, the “VC” stage (i.e. cotyledons unfolded), the V1 stage (appearance of first node and unifoliate leaves), the V2 stage (appearance of the first trifoliate leaf), and thereafter. In certain embodiments, non-destructive genotypic assays are performed in seed using apparati and associated methods as described in U.S. Pat. Nos. 6,959,617; 7,134,351; 7,454,989; 7,502,113; 7,591,101; 7,611,842; and 7,685,768, which are incorporated herein by reference in their entireties. In certain embodiments, non-destructive genotypic assays are performed in seed using apparati and associated methods as described in US Patent Application Publications 20100086963, 20090215060, and 20090025288, which are incorporated herein by reference in their entireties. Published U.S. Patent Applications US 2006/0042527, US 2006/0046244, US 2006/0046264, US 2006/0048247, US 2006/0048248, US 2007/0204366, and US2007/0207485, which are incorporated herein by reference in their entirety, also disclose apparatus and systems for the automated sampling of seeds as well as methods of sampling, testing and bulking seeds. Thus, in a certain embodiments, any of the methods provided herein can comprise screening for markers in individual seeds of a population wherein only seed with at least one genotype of interest is advanced.

### VII. Molecular Assisted Breeding Techniques

Genetic markers that can be used in the practice of the instant invention include, but are not limited to, are Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD), and others known to those skilled in the art. Marker discovery and development in crops provides the initial framework for applications to marker-assisted breeding activities (US Patent Applications 2005/0204780, 2005/0216545, 2005/0218305, and 2006/00504538). The resulting “genetic map” is the representation of the relative position of characterized loci (DNA markers or any other locus for which alleles can be identified) along the chromosomes. The measure of distance on this map is relative to the frequency of crossover events between sister chromatids at meiosis.

As a set, polymorphic markers serve as a useful tool for fingerprinting plants to inform the degree of identity of lines or varieties (U.S. Pat. No. 6,207,367). These markers can form a basis for determining associations with phenotype and can be used to drive genetic gain. The implementation of marker-assisted selection is dependent on the ability to detect underlying genetic differences between individuals.

Certain genetic markers for use in the present invention include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

In another embodiment, markers that include, but are not limited, to single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with aphid resistance loci, regions flanking aphid resistance loci, regions linked to aphid resistance loci, and/or regions that are unlinked to aphid resistance loci can be used in certain embodiments of the instant invention.

In one embodiment, nucleic acid-based analyses for determining the presence or absence of the genetic polymorphism (i.e. for genotyping) can be used for the selection of seeds in a breeding population. A wide variety of genetic markers for the analysis of genetic polymorphisms are available and known to those of skill in the art. The analysis may be used to select for genes, portions of genes, QTL, alleles, or genomic regions (Genotypes) that comprise or are linked to a genetic marker that is linked to or correlated with aphid resistance loci, regions flanking aphid resistance loci, regions linked to aphid resistance loci, and/or regions that are unlinked to aphid resistance loci can be used in certain embodiments of the instant invention.

Herein, nucleic acid analysis methods include, but are not limited to, PCR-based detection methods (for example, TaqMan assays), microarray methods, mass spectrometry-based methods and/or nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. No. 4,683,202; U.S. Pat. No. 4,582,788; and U.S. Pat. No. 4,683,194), using primer pairs that are capable of hybridizing to the proximal sequences that define a polymorphism in its double-stranded form.

Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entireties. However, the compositions and methods of the present invention can be used in conjunction with any polymorphism typing method to type polymorphisms in genomic DNA samples. These genomic DNA samples used include but are not limited to genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. Nos. 6,799,122; 6,913,879; and 6,996,476.

Target nucleic acid sequence can also be detected by probe linking methods as disclosed in U.S. Pat. No. 5,616,464, employing at least one pair of probes having sequences homologous to adjacent portions of the target nucleic acid sequence and having side chains which non-covalently bind to form a stem upon base pairing of the probes to the target nucleic acid sequence. At least one of the side chains has a photoactivatable group which can form a covalent cross-link with the other side chain member of the stem.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283. SBE methods are based on extension of a nucleotide primer that is adjacent to a polymorphism to incorporate a detectable nucleotide residue upon extension of the primer. In certain embodiments, the SBE method uses three synthetic oligonucleotides. Two of the oligonucleotides serve as PCR primers and are complementary to sequence of the locus of genomic DNA which flanks a region containing the polymorphism to be assayed. Following amplification of the region of the genome containing the polymorphism, the PCR product is mixed with the third oligonucleotide (called an extension primer) which is designed to hybridize to the amplified DNA adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, the locus or loci of interest can be directly sequenced using nucleic acid sequencing technologies. Methods for nucleic acid sequencing are known in the art and include technologies provided by 454 Life Sciences™ (Branford, Conn.), Agencourt Bioscience™ (Beverly, Mass.), Applied Biosystems™ (Foster City, Calif.), LI-COR Biosciences™ (Lincoln, Nebr.), NimbleGen Systems™ (Madison, Wis.), Illumina™ (San Diego, Calif.), and VisiGen Biotechnologies™ (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays, as reviewed by R.F. Service (Science 2006 311:1544-1546).

The markers to be used in the methods of the present invention should preferably be diagnostic of origin in order for inferences to be made about subsequent populations. Experience to date suggests that SNP markers may be ideal for mapping because the likelihood that a particular SNP allele is derived from independent origins in the extant populations of a particular species is very low. As such, SNP markers appear to be useful for tracking and assisting introgression of QTLs, particularly in the case of genotypes.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Mapping Aphid Resistance to Linkage Group J

To map a putative QTL for aphid resistance, an aphid resistant line PI594427C was crossed with a susceptible parent AG3602. This mapping population was developed to map a QTL linked to aphid resistance. The mapping population which was evaluated was an F2-derived F4 (F2:F4) population of 173 plants and it was evaluated for an aphid resistance phenotype in large, enclosed cages at six locations. Three aphid nymphs were placed per plant and aphid density was rated at three (3), four (4), and five (5) weeks after inoculation. One repetition was also performed in the greenhouse. The aphid rating was on a scale of 0-4 as discussed in Table 3. As a result four aphid resistance loci were identified (Table 5).

TABLE 5
Genetic positions and LOD scores for putative aphid resistance loci

| Marker | Linkage Group | | |
|---|---|---|---|
| Designation | (LG) | Position (cM) | LOD SCORE (LOD) |
| NS0202737 | J | 52.6 | 19.004 |
| NS0262844 | M | 31.6 | 2.194 |
| NS0100551 | F | 156.2 | 1.868 |
| NS0262972 | K | 98.1 | 1.115 |

The phenotype data from week 4 evaluation was used for the QTL mapping studies (Table 6). At week four (4) after inoculation, the phenotyping data was reported for 173 F2:4 PI594427C×AG3602 populations and recorded (Table 6). The average disease index rating for the aphid resistant parent, PI594427C was 37, and the aphid susceptible parent AG3602 was 79.

TABLE 6
Phenotype of F2:4 PI594427C × AG3602 populations reported as
Disease Index (DI)

| Aphid Rating Disease Index (DI) | Number of Plants |
|---|---|
| >50 | 23 |
| 50-55 | 27 |
| 56-60 | 26 |
| 60-65 | 22 |
| 65-70 | 15 |
| 70-75 | 9 |
| 76-80 | 13 |
| Total | 134 |

Each population was genotyped with 2722 SNP markers across the genome. Single marker and marker regression analyses were performed to determine QTL conditioning aphid resistance. The results yielded 153 informative markers across the genome. A LOD score of significance (LOD=19) was obtained for marker NS0202737 on linkage group J (LG J). A marker analysis was performed using a t-test and NS0202737 had a p-value≦0.01.

### Example 2

### High Resolution Sequencing of the Aphid Resistant Locus on Linkage Group J Using Sequence Capture

Fine mapping provides the greatest ability to compartmentalize variation responsible for soybean phenotypic traits of interest, especially those associated with disease resistance. The ability to identify the causative mutation, a tight disease resistance haplotype window, or a <1 kB linked marker provides the ability for robust deployment of marker assisted selection (MAS) or phenotypic prediction.

The methodology for fine mapping aphid resistance is described in published patent application WO/2011/090987 (incorporated herein by reference in its entirety). In brief, the method comprises screening a population of plants for aphid resistance, and separating plants from the population into at least two subpopulations of plants that are segregating for aphid resistance. DNA from one or more plants in each of the subpopulations of plants is isolated and pooled, and each set of pooled DNA was sequenced to determine the sequence of a plurality of nucleic acids for the genome of each pool from each of the subpopulations of plants. Finally, one or more polymorphisms linked to one or more genes controlling the selected aphid resistance phenotype were identified in the genome of each pool. To fine map aphid resistance, a F2:4 PI594427C×AG3602 mapping population was phenotyped using the methodology described in Example 1 and recombinant pools were generated.

Fine genetic mapping of the genomic region around NS0202737 was performed to further identify SNPs diagnostic for aphid resistance in a breeding program. The F2:4 PI594427C×AG3602 segregating population was used for the aphid resistant trait. The F2 families will either be uniform for the presence of the aphid resistant trait, absent of the aphid resistant trait, or still segregating for the trait. DNA was extracted from each F2 family homozygous negative and homozygous positive. DNA is then pooled in equimolar amounts from each homozygous negative F2 family and each homozygous positive F2 family. The user now has in hand two pools of DNA; one exclusively from homozygous negative aphid resistant plants and one from homozygous positive aphid resistant plants. These pools contain the sum of the recombination events of two meiotic events by the number of individuals sampled.

The resulting sequencing reads were mapped back to the genome and SNPs are called within both the positive and negative libraries. Also, the frequency of these SNPs is calculated within each library. Using this methodology, a population of SNPs were recovered around marker NS0202737 and reported in Table 2 (earlier Table in Specification). Markers identified in this manner can provide for selection of a genomic region around the NS0202737 marker for the aphid resistance trait.

### Example 3

### Shared Haplotypes of Soybean Lines with PI594427C

The shared haplotypes to of 70 soybean lines were evaluated across the soybean genome. Twenty four lines (24) had a rating of <3.5. Four six (46) lines had a rating of <4. The results of the shared haplotypes with PI594427C are showed in Table 7.

TABLE 7
Shared haplotypes with PI594427C

| | | | | N of lines with rating | N of lines with |
|---|---|---|---|---|---|
| | | Hap in | | ≦3.5 | rating ≦4 |
| | Chr | PI594427C | Pos | total N = 24 | total N = 46 |
| N of lines | 1 | GC | 41-8-41.9 | 24 | 46 |
| having the | | GAT | 55.3-55.7 | 22 | 42 |
| same hap | 2 | TA | 90.1-90.2 | 16 | 38 |
| as | | CG | 107.2 | 16 | 38 |
| PI594427C | 7 | TCA | 71.7-72 | 24 | 44 |
| | | CCAG | 75-75.5 | 20 | 36 |
| | | GACT | 97.7-98 | 20 | 40 |
| | | CG | 105.3-105.6 | 18 | 38 |
| | | CT | 106-106.1 | 18 | 38 |
| | 11 | AT | 39.8 | 22 | 42 |
| | | TGA | 40.4-40.5 | 22 | 42 |
| | | AAC | 40.8-40.9 | 22 | 42 |
| | | G | 41.2 | 22 | 42 |
| | | AA | 44.4-44.5 | 22 | 42 |
| | | C | 72.9 | 22 | 42 |
| | 13 | GATA | 129-129.5 | 16 | 26 |
| | | GCCCACG | 180.4-181 | 20 | 32 |
| | 16 | GCGA | 35.0-35.3 | 14 | 20 |
| | | G | 60.3 | 20 | 42 |
| | | T | 183.2 | 18 | 38 |

### Example 4

### Exemplary Marker Assays for Detecting Polymorphisms

In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means. Exemplary primers and probes for amplifying and detecting genomic regions associated with a aphid resistance phenotype are given in Table 7.

TABLE 8
Exemplary Assays for Detecting Polymorphisms

| | | | | | SEQ | SEQ |
|---|---|---|---|---|---|---|
| | Marker | SNP | SEQ ID | SEQ ID | ID | ID |
| Marker or | SEQ | Posi- | Forward | Reverse | Probe | Probe |
| Locus Name | ID | tion | Primer | Primer | 1 | 2 |
| NGMAX007666919 | 16 | 101 | 63 | 64 | 65 | 66 |
| NGMAX007666921 | 17 | 101 | 67 | 68 | 69 | 70 |
| NGMAX008369613 | 23 | 101 | 71 | 72 | 73 | 74 |
| NGMAX008369615 | 28 | 101 | 75 | 76 | 77 | 78 |
| NS0202737 | 35 | 347 | 79 | 80 | 81 | 82 |

### Example 6

### Summary Table of Nucleic Acid Sequences

TABLE 9
Nucleotide Sequences

| Marker or | SEQ ID | Sequence |
|---|---|---|
| Locus Name | NO: | (DNA; 5′ to 3′) |
| NGMAX007664762 | 1 | GTCCAGTACATACGCGTTTCCAAGATATGCTTTTCATTTATAAAAATAG |
| | | CAAACTTACAGTTGTTTGTAATATGCAGGCTTTAATATATTGTCATTGA |
| | | AAHTAGTCTCTACTTAAATTGTGTTTTTAATTTCTGAAATTACATAGAA |
| | | AACATATTCCTAGTTATTGACAGGGGACTAAAACTAAATTATATATACA |
| | | CACAC |
| NGMAX007664838 | 2 | GGAAATCTATGTGGGCACGAACATCTTCCTTTACTCATTTACACCATTC |
| | | CTATTTGGGAACTGATTTTTTTTTTTTAAAAAAAAAGCTGAATCAGGCA |
| | | AAHTTTGAAAATGATTTTTCAGTTTTACTTGTGTTTGTTTGAGATTTTA |
| | | CGTTTGTCCCTAATATGGTCGTTTTATGTAACACTCAATTGTTTGAATT |
| | | TATTA |
| NGMAX007664836 | 3 | TTTACTCATTTACACCATTCCTATTTGGGAACTGATTTTTTTTTTTTAA |
| | | AAAAAAAGCTGAATCAGGCAAATTTTGAAAATGATTTTTCAGTTTTACT |
| | | TGHGTTTGTTTGAGATTTTACGTTTGTCCCTAATATGGTCGTTTTATGT |
| | | AACACTCAATTGTTTGAATTTATTATGGCGTAATGGTTCAACCAATAAC |
| | | GTCCT |
| NGMAX007665386 | 4 | ACATTTAACAACTTTTTAACAAGTCGAACTTATTTAAGTTAGTCAAGCA |
| | | TAATTCAATGAAAGATAATAAGATCCTTGTGTTATGATTTTTGGAGTTA |
| | | GTHGTAGATAATATTGTGATGGTGATTTAAAGATTACAACATAAATTGG |
| | | ACCATGTAAATAAATAGATTTTAGCTATGTTAAATCTTCAAAACAATTT |
| | | CTCT |
| NGMAX007665387 | 5 | CTTTTTAACAAGTCGAACTTATTTAAGTTAGTCAAGCATAATTCAATGA |
| | | AAGATAATAAGATCCTTGTGTTATGATTTTTGGAGTTAGTAGTAGATAA |
| | | TADTGTGATGGTGATTTAAAGATTACAACATAAATTGGACCATGTAAAT |
| | | AAATAGATTTTAGCCTATGTTAAATCTTCAAAACAATTTCTCTTTATTT |
| | | TTATA |
| NS0125096 | 6 | ggccagcttgcatgcctgcaggagaagtatcgcaaaacttaagagtgaa |
| | | ggaaaaagcagtgttgttcagtttttcaccttctcttattttaagccta |
| | | gctttcacataattaacttttctttccaaaactcaagtttahgatttga |
| | | aaaactatgtcctattgatgattataagaccatatagattttcttgcct |
| | | agaacagtggtcaataatttggaactatagtgactttgctgntgtataa |
| | | atttatatttaatatagaattatcaattttcttattgcatctcaaatct |
| | | caatgcctacctattcctcctcatgcaggacatgaagcggcaatgtgat |
| | | gaaaaaaggtttgtatttacacaactgctattgttttttctcatcatta |
| | | tgtgatgtttcctgaatctgttttattgccttcagagatgtttatgaat |
| | | acatgattgcccaacagaaagagaaaggaaagtcaaaaagtgctaaagg |
| | | tgaaagtttcacgctacagcagttgcaagcagacatgctgaatatgaag |
| | | acgaagcaaaactttgtgcctttcggttaaaatcgctgaagcaaggcca |
| | | gtcacgcagtctcctaacacaagcagcgcgtcaccatgctgctcaggtt |
| | | ttcttgaactatgacctttctcattcaaatcttttatcatttcttcagc |
| | | ctagtcttgatgcatctttgttcttgttcttctttcaatatagttgaat |
| | | ttcttccggaaaggacttaaatcactagaggctgttgacccacatgtta |
| | | ggatgattgctgaacgacaacatattgattaccaattcagtggcctgga |
| | | ggatgatggaggtgaaaatgataataatgatgatgggaatgattttgac |
| | | gtcattgaaggtggtgagttgagttgagttttgactacagggcaaataa |
| | | gcaagggccatatattgtttccacatcaccgaactcagcagaggtagga |
| | | aatttgtattactcaaaacttgaatggttttcaaagcctgggtgcatat |
| | | tgaactttattcttattgctcatttgcttttttatttaaaatattacca |
| | | aacttgtcacagttgtcattttatacttgttgcagtctatagctcgcta |
| | | caaattaaaaacattccattgttgattcataatctgaactataaattca |
| | | tcttatcataatcattggcaatgattgccaggtggaagaatcaggccgt |
| | | tcatatattcgagcttcaaccccaga |
| NGMAX007665590 | 7 | AATACTATCCGGAGAAAGTTTGATAGAGAACTTATCACTAAGCATTCTC |
| | | AAACCCTTTTTTTAATCTAAAAACAAACTTTTATTTATCTTGTACTGTA |
| | | GADATTCTTAATCTAGAGGAAAGTTTTATTTGTAATAATTAAGTTTGTT |
| | | AAAAAATGAAGATGAGATAAGGAACCAATTTTTTTTTCTCTTTGGTCAT |
| | | CATAA |
| NGMAX007665668 | 8 | TTTAACTACTCATTATTTTACCTTTAAAATTTTGTGAACATATTTATTT |
| | | AACTTTCCCATTTTCTCCTCTCTTATTTCCTATTTGACTTTATTAAATG |
| | | ATBGTTCCAAGGTAAACACGCCATAGTTAACCTTCTTATAATATATGTG |
| | | GGACTCAATCAAGTGGGATCAATAACACTAAGGATACAAATTGTATCTT |
| | | AGGAA |
| NGMAX007665986 | 9 | tttgatgcctcctctctcatgtggctgaagccattgcttttgggtcaag |
| | | ttattttttaatatgattgccaaagattagtacaagcatggaagaaaca |
| | | agbtagctgtgggtgataatagatattttgaaggaattgtttctgattg |
| | | cctgattttggtagattacaattcccagcaagatcttgtttttgcccat |
| | | agaac |
| NGMAX007666264 | 10 | TTTTGTATCTGGGAAAAAAAACATTCAATCAGGGATGCATGCATTGTCT |
| | | CACACACTGACACATAACGATGGCATCCATGGTGTTGCTGCAGCCTGCA |
| | | GCHGAGCTGGATTTACACAAAAATGAGACCTAGATGAAACGGGTTTCTT |
| | | ATACCAAAAATGAGGCACCAATGCTACAAAGTACAAACAAACTAATATA |
| | | TTGCA |
| NGMAX007666309 | 11 | GTCCATTTGTATGTGTCTGTTCACTAGTAATGGCTGCAGTTGGGGGTAT |
| | | TGTATCTATTCAGTGAAAAGAAAGTTATTAGCAAAACCCCAAAATGGTT |
| | | TABTGAAGTTTAGTATTATGGCCAGAGAGAATCTGGCTTAAATGGGGTG |
| | | AGGAGGAAAACAAATTAAGCTAATGCCATTTCTCTTCATTCTTCAATTT |
| | | TCTAG |
| NGMAX007666777 | 12 | CTTCATGGGTATTGGTATAGTCCCTCATGGCCTCTTGTTCATCGTTTTT |
| | | TCTATTTTTTAATAATACTTTGGGCTAGAGTAAAGGATTGATAGGATGT |
| | | GGDGTGTCATCATAGTTGGGATGTCATTGTTCTGTTTTAATGCTTTTTA |
| | | GGCCATAATTTTCATTGAAAAAATATACACGCGGGTGTGCACATACCCA |
| | | TGCAC |
| NGMAX007666843 | 13 | TATATATATTGACAAAATAGAGGATTGAACCAAAATTGTGAATTGGAA |
| | | AAACAACGAGAATCAAAATTATATTTAAGCCTTTATCTGTTCAAATGTT |
| | | AAGHATGTTCGACCGTGTGATAGAGAGTTGACACTATGATACTCTACTC |
| | | TTAAGAAAATACCATAAAAACTATATAAGAAACCTACATAGAAGATAAA |
| | | ATAACA |
| NGMAX007666844 | 14 | taaaaactatataagaaacctacatagaagataaaataacaactcata |
| | | tatttttattgatgctaatagataaaggaaaatacatttaaatagtcct |
| | | aaahgatattaattcacagataatacttatccaggtgtcaacatcatgt |
| | | tattctaaacatatttaaactaaaattataagaaaaaatatttaactcc |
| | | acccaa |
| NGMAX007666869 | 15 | AAGTGATCTAGGGTAGTGATATAGTAGGAAACATACCGACACGATCAA |
| | | GAAAGCTAAGAGAGGAAGATACACACAATTCCTCAATCTCAATGCTCTC |
| | | ATCHTTCCAAATGCAAATGATGGTTTTATGGCCTTTAGGCCTAACTTAA |
| | | TCCCAGCCTGAAAGTTAGGTCTTGTGTTTATTTAAGTGCTATTGAAAAA |
| | | GAAGCC |
| NGMAX007666919 | 16 | agtcacgcatgatatgcattcttcactgtaaacaaattatcattcgta |
| | | tgtttccaagctggacaatcctaattaaaaaataaaaatagaaaaaaga |
| | | atcgbaagtaattaacaaccctaattagtccaagataaaaaaaagagat |
| | | ggaatgtaaattttttttgtcagattcgtgtcattgttacagttttcaa |
| | | agtgaa |
| NGMAX007666921 | 17 | tgcattcttcactgtaaacaaattatcattcgtatgtttccaagctgg |
| | | acaatcctaattaaaaaataaaaatagaaaaaagaatcgaaagtaatta |
| | | acahccctaattagtccaagataaaaaaaagagatggaatgtaaatttt |
| | | ttttgtcagattcgtgtcattgttacagttttcaaagtgaaaaataact |
| | | aattta |
| NGMAX007666976 | 18 | AACTCGTGCGTGCACCCCGAACACCTGAAAAAGCAATAATAATATAAA |
| | | TAAATTGAATTTTTAAAAAGGAAAAAAAAATGAGAGTAAGTAAAGGAGG |
| | | AGCDTACGATTGGAAGTGCGGACAGTGAAGTGCGCATGTTAGCGGCGGG |
| | | GACTGTGGAATTGAAGAAATAAGCGTCGGCGCCGGCGTCGGCGTCGGTG |
| | | TCGGCG |
| NGMAX007666977 | 19 | TAAGCGTCGGCGCCGGCGTCGGCGTCGGTGTCGGCGGAGGGAGGGCGG |
| | | TGGATGCGGTGGCGCGGCAGGAGAAGAGAGAGAACGACGGCGGAGCTGA |
| | | CACBAGACGGAGTGTCATTTTTCCGGTACGACGTGGGAATGGATAATAA |
| | | TTACAAATTATCTTTATAAGTAGTTCCTATAATATCTGTAGCGGAGTTG |
| | | TGGGAA |
| NGMAX007667014 | 20 | cactcacgatgagtgatgctaactgctatttgttttggttgggtcatt |
| | | tcctactacatcattcgcagagaagagcgaatgacatttaaaaatcaaa |
| | | tgavccgaatgattcgatcggtcagtactcagtatgactgtatgagttg |
| | | gctaaatctcttatccaattttcatagatcattatttcatttaattatg |
| | | gttta |
| NGMAX007667071 | 21 | CTCCCCCGGCGCAGCTCAGCTTACATGCATTATCAGGCCATTCAGCGC |
| | | CTGAAACCTTACGTCTGAAGGGGGCCATTAATGAGCTTCACGTTAATAT |
| | | CCTHATTGATGGAGGTAGTACGCACAACTTCCTCCACCACAGGGTTGCG |
| | | ACGGTGTTAGGTTTTCACCCAAAGAGATAGCTCCACTTAGAGTTACGGT |
| | | GGGTA |
| NGMAX007667072 | 22 | GCTTCACGTTAATATCCTTATTGATGGAGGTAGTACGCACAACTTCCT |
| | | CCACCACAGGGTTGCGACGGTGTTAGGTCTTTCACCCAAAGAGATAGCT |
| | | CCAHTTAGAGTTACGGTGGGTAACGGAGACGAAATCCGTTGTCATCAGC |
| | | TTTGCATGGCTGTTAAAGTACAAATCCAAAGGTACTTTTTCACGGTTGA |
| | | CTTTCA |
| NGMAX008369613 | 23 | taacggagacgaaatccgttgtcatcagctttgcatggctgttaaagt |
| | | acaaatccaaaggtactttttcacggttgactttcacatcttaccattg |
| | | tgtdgcgcgtacgtcgtactaggagtagagtggctcaaaacactggctc |
| | | caatgctcacagattacacttcattgaccatgaagttcattactaaagg |
| | | caagct |
| NGMAX007667077 | 24 | GACGAAATCCGTTGTCATCAGCTTTGCATGGCTGTTAAAGTACAAATC |
| | | CAAAGGTACTTTTTCACGGTTGACTTTCACATCTTACCATTGTGTGGCG |
| | | CGTDCGTCGTACTAGGAGTAGAGTGGCTCAAAACACTGGCTCCAATGCT |
| | | CACAGATTACACTTCATTGACCATGAAGTTCATTACTAAAGGCAAGCTA |
| | | GTTGAA |
| NGMAX007667093 | 25 | AATATGGCTTCCCTCTAATTTGTCTTTCATTAAGTTGTTACTCGAAGA |
| | | ATTTCATCAATCACCTGCAGGCGCCCATATGGGAGTGCAAAAGACCTTA |
| | | CATHGTCTACAGGAAAACTTCACTTGGAGTTCAATTCGAGAGTATACAC |
| | | GTGCTTTTATCGCAAGCTGCTTAACCTGTCAATACACAAAGTACGATAA |
| | | CCGGAA |
| NGMAX007667095 | 26 | TCACTTGGAGTTCAATTCGAGAGTATACACGTGCTTTTATCGCAAGCT |
| | | GCTTAACCTGTCAATACACAAAGTACGATAACCGGAAACCAGGGGGCTT |
| | | GCTHTGTCCTCTTCCGGTATCGGCACAACCCTGGGAAGATTTGTCGATG |
| | | GACTTTATCGTAGGGTTGCCAACTTATCGGGGAAATACTTGCATCTTTG |
| | | TCGTGG |
| NGMAX007667203 | 27 | cattattaatatgaaagcaatacaaggaggacaataccggagcagcat |
| | | gtataaactttctatgttcacctattagtctattacggagttatatata |
| | | btagtatcacaatgtataacaagtaacaacagatatcaaactacaacaa |
| | | tagtgcctcaattggtcatatgcaaaacttattgaacatgcttaagagt |
| | | att |
| NGMAX008369615 | 28 | actcaatacaattcattattaatatgtaaagcaatacaaggaggacaa |
| | | taccggagcagcatgtcttaaactttcttttgttcacctattagtctat |
| | | tacbgagttatatatactagtatcacaatgtataacaagtaacaacaga |
| | | tatcaaactacaacaatagtgcctcaattggtcatatgcaaaacttatt |
| | | gaacat |
| NGMAX007667202 | 29 | gttatatatactagtatcacaatgtataacaagtaacaacagatatca |
| | | aactacaacaatagtgcctcaattggtcatatgcaaaacttattgaaca |
| | | tgchtaagagtattcaaatccaaaagtcagcaattataatgatgcgccc |
| | | caattataaatttttttgaaataatttaagtcccgtaagaaaaatagtt |
| | | aattta |
| NGMAX007667293 | 30 | agacgaaaaaataatttctattttctctactatttagtatgacaatca |
| | | agaatcaaattatcactatattttttcttttttctacttcttctttcaa |
| | | gtghagaaaaaccctaaggagttgcggggttttttctaccaattggggt |
| | | cctcccttcaccactcgcatgcggatggtttacaagattcataactatt |
| | | cttctt |
| NGMAX007667292 | 31 | ACTATATTTTTTCTTTTTTCTACTTCTTCTTTCAAGTGCAGAAAAACC |
| | | CTAAGGAGTTGCGGGGTTTTTTCTACCAATTGGGGTCCTCCCTTCACCA |
| | | CTCBCATGCGGATGGTTTACAAGATTCATAACTATTCTTCTTATTAGAG |
| | | GACACTTACCTAATCAACATTTAAATTTGGTTCTACCTAAATTTTTTTG |
| | | GTTTAC |
| NGMAX007667295 | 32 | CACCACTCGCATGCGGATGGTTTACAAGATTCATAACTATTCTTCTTA |
| | | TTAGAGGACACTTACCTAATCAACATTTAAATTTGGTTCTACCTAAATT |
| | | TTTHTGGTTTACTCCAACATTTCAAAAAAAATATCAACAACACATTTAG |
| | | AGGACACTTACCTAGTCAATTATTTTTTCCTATGTTTTTTTAGCTTATT |
| | | TTAATA |
| NGMAX007667294 | 33 | GCATGCGGATGGTTTACAAGATTCATAACTATTCTTCTTATTAGAGGA |
| | | CACTTACCTAATCAACATTTAAATTTGGTTCTACCTAAATTTTTTTGGT |
| | | TTAHTCCAACATTTCAAAAAAAATATCAACAACACATTTAGAGGACACT |
| | | TACCTAGTCAATTATTTTTTCCTATGTTTTTTTAGCTTATTTTAATACT |
| | | ACTAAC |
| NGMAX008383011 | 34 | ACATTTAACAACTTTTTAACAAGTCGAACTTATTTAAGTTAGTCAAGC |
| | | ATAATTCAATGAAAGATAATAAGATCCTTGTGTTATGATTTTTGGAGTT |
| | | AGTDGTAGATAATATTGTGATGGTGATTTAAAGATTACAACATAAATTG |
| | | GACCATGTAAATAAATAGATTTTAGCCTATGTTAAATCTTCAAAACAAT |
| | | TTCTCT |
| NS0202737 | 35 | aaaagtgtatacatgaggattgagggtacatatatttagggatgattta |
| | | ccccatgtacatgtggatcccaattgctaacatgaagatgcacacgaga |
| | | gchaagtagaggatagcatggatcaatattgataatccactggtagaaa |
| | | attaccaaactctatgaacctgcctatccaggtatttggaccaagagcc |
| | | ctggagtgaggagaatgaaaagcaccacagacacaaaaactggccccca |
| | | atctcccatttcctccttctccttaatttctctttgatctcctaattat |
| | | ttctctatctagctatgtgaatggtatagtagttgcaatgacaatgagt |
| | | gcaggaggacttaaaaaggaatggagctgaaaagtggtcacttt |
| NGMAX007668492 | 36 | accttcaaaaatatgaaatattcttcagtaggaacagtcttcaaagct |
| | | cccacaattttgtcggagccaatggtgtctagattgatgctattgaagt |
| | | attdgtttcttaaatgttgcataatttcttaaatgtagcactaatgttg |
| | | caatcttcagggagcaacaacatttttggtgctggttgtgcatacgaaa |
| | | gagtaa |
| NGMAX007668494 | 37 | GTAGCACTAATGTTGCAATCTTCAGGGAGCAACAACATTTTTGGTGCT |
| | | GGTTGTGCATACGAAAGAGTAATGGAGATTTTGTTTTGGTTGAAACATC |
| | | ATGDAAGCATGTTTCCCCACTAGTGTTCGAGGGAGAAACAATAACTTTA |
| | | GTGGATGTCATCACTTGGTTCAAAGAAATTAATCTACATGTTATTATGG |
| | | AATTTG |
| NGMAX007668495 | 38 | tgcatacgaaagagtaatggagattttgttttggttgaaacatcatgg |
| | | aagcatgtttccccactagtgttcgagggagaaacaataactttagtgg |
| | | atghcatcacttggttcaaagaaattaatctacatgttattatggaatt |
| | | tgattgcaagcacattgttgagtgtttagcaaatgatagcacaaatcac |
| | | attgaa |
| NGMAX007668903 | 39 | GTAGCACTAATGTTGCAATCTTCAGGGAGCAACAACATTTTTGGTGCT |
| | | GGTTGTGCATACGAAAGAGTAATGGAGATTTTGTTTTGGTTGAAACATC |
| | | ATGDAAGCATGTTTCCCCACTAGTGTTCGAGGGAGAAACAATAACTTTA |
| | | GTGGATGTCATCACTTGGTTCAAAGAAATTAATCTACATGTTATTATGG |
| | | AATTTG |
| NGMAX007668908 | 40 | attcaaataacagagtggttttcagtagtttctgtagtggctgcaact |
| | | gaggaaaaatacatgtcacaccaacatattgtggccataccctgtagat |
| | | ctchaaatagagtagtagagttccatagaaaaacattctgtaattgttt |
| | | gattaaaagataggataaatacttagtccttgcaatttagctttttttt |
| | | tcatc |
| NGMAX007669116 | 41 | TGCCATGTTATTAATCTCAAACAATGATACCTATTATTATTTGAGTGA |
| | | TCAATTCCAATCTATCCAAATTTCTTAATTTGGAAGATTTCACTGCATA |
| | | ACAVTCTCAAAAGTGTATTCTTAATTTGGAAGATTTCACTGCATATATA |
| | | CAGATTTTTCACCTAAAAAGGTGTCTTATTAGTAATATTTTTCTTATTA |
| | | AAAAAG |
| NS0122151 | 42 | gtttttcctcactctctccatcatgttcatgtcaccactctccaagtag |
| | | ttactcccttgcahgtcatgctaacttggagagctgattgcatgcttct |
| | | ctgtaacataatcctagtgtacaccttaataggatgggtttcaattatt |
| | | cagttgttganaagtcattactactcagctaggaaaggcaggcatggaa |
| | | tggccattttctaaataatttgttataacaattgaagagagtgataaca |
| | | gggtaagaagtgagtgaaagctacagcctacacaaaagagagaacttac |
| | | tttgaaaggaatttataaaattgaatcaccaaatccaggtcattctcat |
| | | ataccgtactgagtatcccaggcatgagatgccaaatcttgggtctgtt |
| | | gnataaattatattaataacaatgtttcagaataaaatactatgaagtt |
| | | tggttatacaaatacaatagaacagattctgcatgcaaccattccatgt |
| | | atcaaaatgctcaagttaaccccacagctatcctagactatataatggg |
| | | aggaaaagaaatgtanagtaaaataaaagttaagaaaggtcattccttt |
| | | caaatgta |
| NGMAX008369616 | 43 | CAACAAAGCATTTGCTTCTGAAGCAATCTCAGAAGAAGTAGCAAATGC |
| | | ACAGAAGTACATCACAAGAAGCAGCGAAAGTGGTTGGAGCTTCATGGAC |
| | | AGADGAGTTGGAAATGTGAACACCATGAGGAATTTTTTAGGAAAGTTTT |
| | | GATCACCGATTGTCTGATACAATCAAATGAATAGGAGAAGTACTTGCAT |
| | | GCAATT |
| NGMAX008369614 | 44 | CTCATCTAAGTAAACCTTATCAAGATGTACTTTCTTTTCCTCGAGAAC |
| | | ATCTCAGATCCAATGATACCTTACGTCAAGGTGCACATCCTTGAACATG |
| | | TCTHCATCATGAGCGATGTTATCCTCTATCTTGAACTGCACGAGCTAGT |
| | | GACGGCTCCGGATGAACCTGACTTTTAATACCACTGTTGGGAAAAACTC |
| | | GATGGG |
| NGMAX007670330 | 45 | TACTACGTTGGTATTGTCATAAGCACGACGTAGAAAGCACGGGTTTCA |
| | | ACTACGGTACTAACCTAAGCACGACATAGAAAGAACAAGTATTGTCATC |
| | | GTCVCTCATGTCAACAACGTCTTCGAAAGCTTACGTTCAATTACTGTCG |
| | | TGGCTGACCCCGTCGCAGGATTCAGTGCTTTCTAAAACGGTGTGTTGCG |
| | | ACCGTC |
| NGMAX008369617 | 46 | CCTTAAGAAGGATTCTCAAAAGTTTACTTTTAGCTCCAACAAGACATGT |
| | | TCTTACATCTAAGCCCAACCAAACAAAAATAGAAAAACCAAATTTTAAA |
| | | TTHTTTATTATCAACCTCATGATCACCATGTCTACCACGATTTATCCAT |
| | | GGTTGTGTTTGGTTATCAATTTTAGCTTTTTCATCAATTTTGGTTAATA |
| | | ATTTT |
| NGMAX008369618 | 47 | AAGTTTACTTTTAGCTCCAACAAGACATGTTCTTACATCTAAGCCCAA |
| | | CCAAACAAAAATAGAAAAACCAAATTTTAAATTTTTTATTATCAACCTC |
| | | ATGVTCACCATGTCTACCACGATTTATCCATGGTTGTGTTTGGTTATCA |
| | | ATTTTAGCTTTTTCATCAATTTTGGTTAATAATTTTGATTCAAATTCAA |
| | | TCGTCA |
| NGMAX007670824 | 48 | TATTTTGTTATAAGATTTTATCTCATACTTATACTTTAATAAAAAAAA |
| | | TATTAAAAATAATTAATCAAATTTCATGATAGATATTAATCATCATCAA |
| | | ACTVGATAAGTAGAATTGGTTCACTAACCCAATTAAAAACAGGCCCTAT |
| | | GATATTGCCCCGTTAAGAAAAATCAATGGGGGCCAAATAGTATTGTGTT |
| | | TTTTAA |
| NGMAX007671248 | 49 | TAGATTACTTCTTGATTATTGAAGTAGAGTATCTTATGGATGGTTATT |
| | | ATCCCAAGCCAATTATTTGTGATACTTACTTGACAAGGGTCACATGATT |
| | | GAGBCTAAAGCTTTTACACTTTTGTGATTGGGGAGTGCAAGCTCTCCAA |
| | | GTGTTGTGTTGATTACATGGAAATTCCTAGGGATGACAAAAAAATTGAA |
| | | GATATC |
| NGMAX007671280 | 50 | GGATTGCTTTTATGTAATTATAGTAATTAGGCATATTTTGCAAATCCT |
| | | TAATTATGACTCCCTCTATAGGTAACATCTTTTGGTGAGTGATTCACAT |
| | | ACAHGAATGGTTCCCTCGATGTCTTTTCTTAACAATTATTGAATTACTT |
| | | AATGTGAAGTGTGAATTTTAACAAATTAATTATAGTCGTGAAATATTTT |
| | | CAAGTT |
| NGMAX007671348 | 51 | TGAAGTTGGCAGCCTCCTTGGCAGGATCATCCTTAGAGTTTGAGGCAA |
| | | CAAAACCACGCTTGAGATCCTTGACTGCAACATTCTTCACATTAAATCC |
| | | TACVTTGTCACCTGGAAGGGCCTCCTGGAGAGCTTCATGGTGCATCTCC |
| | | ACAGACTTAACTTCAGTTGTCAGGCCAGTGGGAGCAAAAGTCACCACCA |
| | | TACCGG |
| NGMAX007671426 | 52 | AGTTTCCCGATCAACTCTACTATAAAAAGAGGTTCATGTATAACCAAA |
| | | TGTTGTTGGATCCTTAATCCTACAGTTTAGGTAAACAATTATGCATTGT |
| | | GACDGTCATTTAAGATTAAGCCCAATTAAAAGTGATCTAATAAGGTTTA |
| | | AGAAATTTGGGCTTTGATGTATCTAATAGGGTATAAACCTTTATGTATA |
| | | GAGACC |
| NGMAX007671425 | 53 | GACATCCCATGAGTAGAGAAAGGTTGAGAGAAAAACTAACTCAAGAGA |
| | | GTACGTGTCAACCTAAAAGACCAAATTGCACAACTTCAAAGAGCGATTG |
| | | ATCVTCATGAATTCAGGTATGCTTTCATTTCAATTTCTTAATGGTTAAA |
| | | ATGATTGATTCGAGATTGATAGATGACAGATTTAATTTAAGGGAGCGTT |
| | | TGGTAG |
| Satt686 | 54 | ACGGAAAATAAATGAAACTAAGAAAATAAAATTGAAATTTTATGACAAC |
| | | TAAAAACAAAATAATAATAATAATAATAATAATAATAATAATAATAATA |
| | | ATATACAATAGAAGCCAAACCATACTCCAAAACAAGAAAACATCAATTA |
| | | GAGTAATTGTTAGTTAATGCAGTTAGCTTAGTCAACAAAAAGCTCCTGT |
| | | TCCTGTAACCAAAATATATACCCTGCCTAAAGACAACATAACATAGACA |
| | | CTTGAATCAAATTCTTCTGCTTCTCTATCTGATAGAGT |
| NS0115450 | 55 | attttaccctgaggttttgccatatggtgaaactttcttccatgagcca |
| | | gttggtagagcttctgatggtcgccttatcatagatttcattggtatat |
| | | accttttcaataattctcatcatttttttgctttatttcttattcattt |
| | | atttgtgaaacacgatcaaatgacatgtttgattttctatctactttgt |
| | | tttctattctttaatcatcatgtggtgtctcacaacacaagctaaatct |
| | | tcttttctttgagttttgttccacaaagattggattccagtttgtgttg |
| | | ttattggagttataagatacatgtggttgaagggagaagagggaagggt |
| | | gatggaggtcatgagttagaatattttttattaacaaaaattaacaaat |
| | | taacaactaatatatattgachgataaaaagaattgtgttgtcattgac |
| | | taagtgaccaacacaaacatctctacccaaatgaataaagttgggattc |
| | | aataagaatgagttgtgaaatttttttacacaatatactgtctaccatt |
| | | atctatttagcagccatagaatcctatgatggtcatacttacaaagtgg |
| | | tacataaaaaatataaagttttcacnaagtgaaaatttaatatgagaag |
| | | atcataatatatatattaagggatctagttcaattngttgaagtgtata |
| | | taagtattgtaaatttcctcttatcgtgttcaatttctgcatatcaaaa |
| | | aatatatcataatatatatagttctattgccaagaaactatcatactag |
| | | tgatttggtgccatttgtgatgcagcccagcatctaggtttcccattat |
| | | gagtgcatacataaattcaattgggacaagttataggcacggtgcaaac |
| | | ttt |
| NS0120948 | 56 | atggatgttatcaataagtgatctctaaaaaggctgttgaggaaatatt |
| | | atatactcgtaagttgtattgagttacactattgaaaatgtctgattah |
| | | attcttcagcatgatcaaggatgaacttttgccatttagcaacctgaat |
| | | tttagcctgctgttttaggaggtggaagctacgagaaaggctgatcctt |
| | | ggatttctgcaactattcaatccttaaagaaagcatctccaacaagcct |
| | | taaaatctttcttagatcggtatgtgtccagaatgtattaaaagttctg |
| | | ttttatgtacattctaaaattcacttctcttctgtgttcaactgttgaa |
| | | gtatcatttttatcatcatcatcattttactatcattattgtgattata |
| | | acnnctgttttttttttaaatttattttggcctttttcagattagacaa |
| | | ggaaggaccaaggtgttggacaatgccttgtttctgattatagagttgt |
| | | ttgtcatattctaaaaggacactacagcaaggatttcttcgaggttatc |
| | | tgatgattcctatacatagttatgcttttaattatttttctttcatgcc |
| | | ctatggttaatgttacccttt |
| NS0119584 | 57 | AGGTGATGCCTTTGGAATTTTTGCTTGTTCTTTCTTTTCTCTTTTAATA |
| | | TTTTACTTTATTCACTGTTCTTAATAGTTCCATGCCTTGTTTATGTATA |
| | | ACTTTTCTATTTTCAGTTGCTGGTTTTGGTTGTTTAAGTAAGGTTGGAC |
| | | ATTAGACATTTCAAAATTGAAACCAATACTCTGATGCATTACATAGCAG |
| | | TTCCTGATTCATCGCATGTTTTAAAAAGTAATTTATGGTGTGTCAAAGC |
| | | CATCATTTTTCTATTTTTATGCATTGTCGAAGAATATGTGGATTCGACT |
| | | TGTCTTATACTGTAGTTAAGAACATTTGTTGTTGTCTAAATAAGATGTA |
| | | TATGCTGTTGTGCTTCTTAAAGCAGCGTATGTTATGCGTTAGTAGAAGG |
| | | TACAAATGGCTGAACGGGCAAAACCTATTATTGTTTTGTTTATGATGAG |
| | | AAATGAGAAATAAATGATGCTGCTTGGTGCTATTTTGTTATTCTTTCCA |
| | | TTGTATTTGATAAACATAAACAGTAAATATGTAAAACATGATACTGTAA |
| | | TTTCTTCAATTGATGGATGHAGTGGACTGCATTCTCATGTATAGAAGCT |
| | | GATATTTGTTTTTTTGTCTTGGGATGTAATTTGCAGGAGGGTGCTGCTG |
| | | CTGTTGCTGTGGAAGAAGCCAAAAAGAGTAATCATCTGCAGGTCGACTC |
| | | TAGAGATAACCCCGGGTACC |
| NS0093252 | 58 | TAAACATGTGACAATATGAAATTAATTAAATTTTATGCAATGAAAAAGA |
| | | AATTTTAAAATATTTTGTACTTTGAAGCAAATTAAATACACTAAACGTG |
| | | TTTATTTTTTAAGAAATTTGACACAAATTAACCAAATAAGCAAATATTA |
| | | GACATGGAAGATATTTAATAAAAAATAAAAAAAATCAAGTAGTCATGAA |
| | | CTCAACATCCAAAACAACTTATACATGAACATGCATGCTGCCTTGAAGG |
| | | CATAACATATCAAAAACATGGCTGTCATATATATAAGGTTAATAAGTTT |
| | | ATATAAACAACAGAATAGGAAGTTTCACTTGDATGAGAACCATTGGGCA |
| | | TATTAGATGGTAGTGTTTCCTGCTTAATAGGTGATTGAATATCATTTCT |
| | | AAAAGTATGAGTTTGCCATATGAACTAAAAGGGTCTCTGATACCTGTAC |
| NS0093989 | 59 | GCATGCCTTGCAGAAATTTAAGAATAAAAGTTTAAACATTTTTGTAACC |
| | | AAATTTCAACATAAAACTAAAAAAAATTAGAAGCAACCACATGTTGCAA |
| | | TTTAAAGTTAAAAATDGTACAGAGATCTATTCCCTACCACAATGTATCT |
| | | GTCTTCAGTAACAGCTAGCATTTCCCACCACGTGATGAAGCCTTTTAAT |
| | | GGCTTCCTTCATCTCCTTCATTTCCCAAATATTAGACATGGAGGATATT |
| | | TAGAAAATAAATAAAAATAAAGTANTCAAGCTGTATGATAAGTAATTAT |
| | | TTATATGTAATTACTTTCTTTCCCAAATTAATTCGATACTAGTAATCTT |
| | | TACTTAAAAGGCTTTAATAAATATATAATAATTAATAAAAATATATATA |
| | | CTTATTAATAAAATATACTGTGAGTAATAATACAATCTTAAGAATATTA |
| | | TCTAAGGTAGAA |
| Satt280 | 60 | TCTCTAACAAAGGGCAAGGGATAAACAACAAATGGCACAACATAGACGG |
| | | CGACAAAGCCGATGACAACGACAGAGACAAAGGTTCCGGTGGAGAGAAT |
| | | GGCACCGAAGCCGATGCGGCATTGGTCGGCGCGGAGGGCAATGAGGGGG |
| | | AAGATGTCGAGGGTGCTGTTGCC |
| NS0203255 | 61 | AGCCTGACCTACATGTTGGTGTGGTTGGTCTCVGTGGACTAGGTCACAT |
| | | GGCTGTCAAGTTTGCCAAAGCTTTTGGAGCTAAGGTCATAGTAATAAGT |
| | | ACATCACCTAGTAAAAAGGATGAAGCAATACAACATCTTGGAGCTGATT |
| | | CCTTTCTACTAAACCGTGACCAAGATCAGATGCAGGCAACTATATATAT |
| | | GCCAATGCCATGCATTTGGAATATTGTGAAAATATCAAAATACACACAT |
| | | TTCTCTCAATTAACTAATGTTGATAATTTCGTAGGGTACAATGGGTGCT |
| | | TTGGATGGTATTATTGACACAGTTTCTGCGGTTCATCCTCTCTTACCTC |
| | | TCATTGGTTTGCTCAAGTCTCATGGAAAGATTGTAATGGTGGATGCACC |
| | | AGAGAGGCCTCTAGAACTACCAGTCTTTCCTTTACTTGCTGGTAAGCAT |
| | | GTGAAAAATATTTCCCTTTATAGGATGCAGAGCACATGAAAGCATATGT |
| | | GATGATTCATATATAAGAATAGTTTAGTGCAAAACTTAAGTAGATTGAT |
| | | GCCAATTAACTTAGGTACTATTTAACATAATTGGCAAGGAGAAAGATAG |
| | | TTGCTGGCACTCTGATTG |
| Satt529 | 62 | GCACAATGACAATCACATACAATGAGCGAAATTAGTTTCT |
| | | GACCCAGTGTGTTAGGGACATCTGTGATCTCTGATCCAGA |
| | | ATCAATTTTTTTCCACTCAATATTAAAAAAATGAAAAGTG |
| | | CATTAAGAAACATTATTATTATTATTATTATTATTATTAT |
| | | TATTATTATT TTCGTCCTGATAATTTATCCTTTTTTATG |
| | | CCTTAATGTGA |
| NGMAX007666919-F | 63 | tgtttccaagctggacaatcctaatt |
| NGMAX007666919-R | 64 | ggtcttataaagcatcaaagaggacat |
| NGMAX007666919-P1 | 65 | actcaagtttaagatttgaa |
| NGMAX007666919-P2 | 66 | aactcaagtttatgatttg |
| NGMAX007666921-F | 67 | gtttccaagctggacaatcctaattaaaaaat |
| NGMAX007666921-R | 68 | accacttttcagctccattcctttt |
| NGMAX007666921-P1 | 69 | cctcctgcactcatt |
| NGMAX007666921-P2 | 70 | cctcctccactcatt |
| NGMAX008369613-F | 71 | tgttcatgtcaccactctccaagta |
| NGMAX008369613-R | 72 | gcatgcaatcagctctccaa |
| NGMAX008369613-P1 | 73 | cttgcaagtcatgcta |
| NGMAX008369613-P2 | 74 | tcccttgcatgtcat |
| NGMAX008369615-F | 75 | ggagcagcatgtcttaaactttctt |
| NGMAX008369615-R | 76 | agtttgatatctgttgttacttgttatacattgtga |
| NGMAX008369615-P1 | 77 | ctattagtctattacggagttat |
| NGMAX008369615-P2 | 78 | cctattagtctattactgagttat |
| NS0202737-F | 79 | cctccttctccttaatttctctttgatctc |
| NS0202737-R | 80 | accacttttcagctccattcctttt |
| NS0202737-P1 | 81 | cctcctgcactcatt |
| NS0202737-P2 | 82 | cctcctccactcatt |

Polymorphic nucleotide bases are designated in Table 8 according to the WIPO Standard ST.25 (1998), Table 1, as follows: r=g or a (purine); y=t/u or c (pyrimidine); m=a or c; (amino); k=g or t/u (keto); s=g or c (strong interactions 3H-bonds); w=a or t/u (weak interactions 2H-bonds); b=g or c or t/u (not a); d=a or g or t/u (not c); h=a or c or t/u (not g); v=a or g or c (not t, not u); and n=a or g or c or t/u (unknown, or other; any.)

Having illustrated and described the principles of the present invention, it should be apparent to persons skilled in the art that the invention can be modified in arrangement and detail without departing from such principles.

Although the materials and methods of this invention have been described in terms of various embodiments and illustrative examples, it will be apparent to those of skill in the art that variations can be applied to the materials and methods described herein without departing from the concept, spirit and scope of the invention. All such similar substitutes and modifications apparent to those skilled in the art are deemed to be within the spirit, scope and concept of the invention as defined by the appended claims.

We claim:

1. A method of identifying a soybean plant that comprises a genotype associated with an aphid resistance phenotype, comprising:
i) detecting in said soybean plant an allele in at least one aphid resistance marker locus associated with the aphid resistance phenotype wherein the aphid resistance marker locus is in a linkage group J genomic region flanked by:
a) loci NGMAX007665986 and NGMAX007668908;
b) loci NGMAX007666844 and loci NGMAX007668908;
c) loci NGMAX008369613 and loci NGMAX007668908;
d) loci NGMAX007667203 and loci NGMAX007668908;
e) loci NGMAX007667293 and NGMAX007668908;
f) loci NGMAX007667294 and NGMAX007668908;
g) loci NGMAX008369613 and NGMAX007668495;
h) loci NGMAX007667203 and NGMAX007668495;
i) loci NGMAX007667293 and NGMAX007668495;
j) loci NGMAX007667294 and NGMAX007668495;
k) loci NGMAX008369613 and NGMAX007668492;
l) loci NGMAX007667203 and NGMAX007668492;
m) loci NGMAX007667293 and NGMAX007668492; or,
n) loci NGMAX007667294 and NGMAX007668492; and,
ii) denoting that said plant comprises a genotype associated with an aphid resistance phenotype.

2. The method of claim 1, wherein said method further comprises the step of selecting said denoted plant from a population of plants.

3. The method of claim 1, wherein said denoted plant does not comprise an allele of a Satt686, Satt280, Satt529, NS0115450 (SEQ ID NO:55), NS0122151 (SEQ ID NO:42), NS0125096 (SEQ ID NO: 6), or NS0120948 (SEQ ID NO:56) marker that is associated with an aphid resistance phenotype.

4. The method of claim 1, wherein said denoted plant does not comprise alleles of the Satt686, Satt280, Satt529, NS0115450 (SEQ ID NO:55), NS0122151 (SEQ ID NO:42), NS0125096 (SEQ ID NO: 6), and NS0120948 (SEQ ID NO:56) markers that are associated with an aphid resistance phenotype.

5. The method of claim 2, wherein said selected plant exhibits an aphid resistance phenotype.

6. The method of claim 1, wherein said genotype associated with an aphid resistance phenotype comprises at least one polymorphic allele of at least one marker selected from the group consisting of NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35).

7. A method for obtaining a soybean plant comprising in its genome at least one aphid resistance locus, compromising the steps of:
i. genotyping a plurality of soybean plants with respect to at least one aphid resistance locus in a linkage group J genomic region flanked by:
a) loci NGMAX007665986 and NGMAX007668908;
b) loci NGMAX007666844 and loci NGMAX007668908;
c) loci NGMAX008369613 and loci NGMAX007668908;
d) loci NGMAX007667203 and loci NGMAX007668908;
e) loci NGMAX007667293 and NGMAX007668908;
f) loci NGMAX007667294 and NGMAX007668908;
g) loci NGMAX008369613 and NGMAX007668495;
h) loci NGMAX007667203 and NGMAX007668495;
i) loci NGMAX007667293 and NGMAX007668495;
j) loci NGMAX007667294 and NGMAX007668495;
k) loci NGMAX008369613 and NGMAX007668492;
l) loci NGMAX007667203 and NGMAX007668492;
m) loci NGMAX007667293 and NGMAX007668492; or,
n) loci NGMAX007667294 and NGMAX007668492;
and,
ii. selecting a soybean plant comprising in its genome at least one aphid resistance locus comprising a genotype associated with an aphid resistance phenotype.

8. The method of claim 7, wherein said selected soybean plant exhibits aphid resistance.

9. The method of claim 7, wherein said selected soybean plant does not comprise an allele of a Satt686, Satt280, Satt529, NS0115450 (SEQ ID NO:55), NS0122151 (SEQ ID NO:42), NS0125096 (SEQ ID NO: 6), or NS0120948 (SEQ ID NO:56) marker that is associated with an aphid resistance phenotype.

10. The method of claim 7, wherein said selected soybean plant does not comprise alleles of the Satt686, Satt280, Satt529, NS0115450 (SEQ ID NO:55), NS0122151 (SEQ ID NO:42), NS0125096 (SEQ ID NO: 6), and NS0120948 (SEQ ID NO:56) markers that are associated with an aphid resistance phenotype.

11. The method of claim 7, further comprising the step of assaying for the presence of at least one additional marker, wherein said additional marker is either linked or unlinked to a linkage group J genomic region flanked by any one of the loci sets of (a), (b), (c), (d), (e), (f), (h), (i), (j), (k), (l), (m), or (n).

12. The method of claim 7, further comprising assaying said selected plant of step (ii) for an aphid resistance phenotype.

13. The method of claim 7, wherein said wherein said aphid resistance locus is genotyped for at least one polymorphic allele of at least one marker selected from the group consisting of NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35).

14. A method for identifying a soybean plant comprising in its genome at least one introgressed aphid resistance locus, the method comprising:
crossing a first soybean plant with a second soybean plant comprising i) an aphid resistance locus in a linkage group J genomic region flanked by:
a) loci NGMAX007665986 and NGMAX007668908;
b) loci NGMAX007666844 and loci NGMAX007668908;
c) loci NGMAX008369613 and loci NGMAX007668908;
d) loci NGMAX007667203 and loci NGMAX007668908;
e) loci NGMAX007667293 and NGMAX007668908;
f) loci NGMAX007667294 and NGMAX007668908;
g) loci NGMAX008369613 and NGMAX007668495;
h) loci NGMAX007667203 and NGMAX007668495;
i) loci NGMAX007667293 and NGMAX007668495;
j) loci NGMAX007667294 and NGMAX007668495;
k) loci NGMAX008369613 and NGMAX007668492;
l) loci NGMAX007667203 and NGMAX007668492;
m) loci NGMAX007667293 and NGMAX007668492; or,
n) loci NGMAX007667294 and NGMAX007668492;
and ii) at least one additional polymorphic locus located outside of said linkage group J region, to obtain a population of soybean plants segregating for the aphid resistance loci and said at least one additional polymorphic locus; and,
detecting said polymorphic nucleic acid in at least one soybean plant from said population of soybean plants, wherein said one soybean plant lacks said additional polymorphic locus, thereby identifying a soybean plant comprising in its genome at least one introgressed aphid resistance locus.

15. The method of claim 14, further comprising the step of selecting said one soybean plant, thereby obtaining a soybean plant comprising in its genome at least one introgressed aphid resistance locus.

16. The method of claim 14, wherein said identified or said selected soybean plant does not comprise an allele of a Satt686, Satt280, Satt529, NS0115450 (SEQ ID NO:55), NS0122151 (SEQ ID NO:42), NS0125096 (SEQ ID NO: 6), or NS0120948 (SEQ ID NO:56) marker that is associated with an aphid resistance phenotype.

17. The method of claim 14, wherein said identified or said selected soybean plant does not comprise alleles of the Satt686, Satt280, Satt529, NS0115450 (SEQ ID NO:55), NS0122151 (SEQ ID NO:42), NS0125096 (SEQ ID NO: 6), and NS0120948 (SEQ ID NO:56) markers that are associated with an aphid resistance phenotype.

18. The method of claim 14, wherein said aphid resistance locus comprises at least one polymorphic allele of at least one marker in a genomic region of said linkage group J region that is flanked by loci NGMAX007667294 and NGMAX007668492.

19. The method of claim 14, wherein said polymorphic nucleic acid detected in step (ii) is detected with at least one marker selected from the group consisting of NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35).

20. The method of claim 4, wherein said identified or said selected plant is aphid resistant.

21. The method of claim 14, wherein said identified or said selected plant is assayed for aphid resistance.

22. The method of claim 14, wherein said additional polymorphic locus is detected with a genotypic marker, a phenotypic marker, or both.

23. The method of claim 14, wherein said additional polymorphic locus is a linked polymorphic locus located on linkage group J but not within said linkage group J genomic region flanked by any one of markers (a)-(m), or (n).

24. The method of claim 22, wherein said linked polymorphic locus is detected with at least one marker that is located within a genomic region of the soybean genome flanked by:
a) NGMAX007664762 and NGMAX007665668; and/or,
b) NGMAX007669116 and SATT529.

25. The method of claim 22, wherein said linked polymorphic locus is detected with at least one marker selected from the group consisting of NGMAX007665590, NGMAX007665668, NGMAX007666264, NGMAX007666309, NGMAX007666777, NGMAX007666843, NGMAX007666869, NGMAX007666919, NGMAX007666921, NGMAX007666976, NGMAX007666977, NGMAX007667014, NGMAX007667071, NGMAX007667072, NGMAX007667077, NGMAX007667093, NGMAX007667095, NGMAX008369615, NGMAX007667202, and NGMAX007668494 and/or with at least one marker selected from the group consisting of NGMAX007669116, NGMAX007668903, NGMAX007668494, NGMAX008369613, NGMAX008383011, or NS0202737.

26. A soybean plant comprising:
i) an aphid resistance locus in a linkage group J region that is flanked by: a) loci NGMAX007665986 and NGMAX007668908; b) loci NGMAX007666844 and loci NGMAX007668908; c) loci NGMAX008369613 and loci NGMAX007668908; d) loci NGMAX007667203 and loci NGMAX007668908; e) loci NGMAX007667293 and NGMAX007668908; f) loci NGMAX007667294 and NGMAX007668908; g) loci NGMAX008369613 and NGMAX007668495; h) loci NGMAX007667203 and NGMAX007668495; i) loci NGMAX007667293 and NGMAX007668495; j) loci NGMAX007667294 and NGMAX007668495; k) loci NGMAX008369613 and NGMAX007668492; l) loci NGMAX007667203 and NGMAX007668492; m) loci NGMAX007667293 and NGMAX007668492; or, n) NGMAX007667294 and NGMAX007668492; and,
ii) one or more polymorphic loci comprising alleles or combinations of alleles that are not found in a aphid resistant soybean varieties harboring said aphid resistance locus, and that are linked to said aphid resistance locus, wherein said soybean plant is aphid resistant.

27. The soybean plant of claim 26, wherein said soybean plant does not comprise an allele of a Satt686, Satt280, Satt529, NS0115450 (SEQ ID NO:55), NS0122151 (SEQ ID NO:42), NS0125096 (SEQ ID NO: 6), or NS0120948 (SEQ ID NO:56) marker that is associated with aphid resistance.

28. The soybean plant of claim 26, wherein said soybean plant does not comprise alleles of the Satt686, Satt280, Satt529, NS0115450 (SEQ ID NO:55), NS0122151 (SEQ ID NO:42), NS0125096 (SEQ ID NO: 6), and NS0120948 (SEQ ID NO:56) markers that are associated with aphid resistance.

29. The aphid resistant soybean plant of claim 26, wherein said aphid resistance locus comprises an introgressed region of the soybean genome that is flanked by loci NGMAX007667294 and NGMAX007668492.

30. The aphid resistant soybean plant of claim 26, wherein said soybean plant comprises an allele of at least one marker selected from the group consisting of NGMAX007666919 (SEQ ID NO: 16), NGMAX007666921 (SEQ ID NO: 17), NGMAX008369613 (SEQ ID NO: 23), NGMAX008369615 (SEQ ID NO: 28), NGMAX007667202 (SEQ ID: 29), NGMAX008383011 (SEQ ID: 34), and NS0202737 (SEQ ID NO: 35) that is associated with aphid resistance.

31. The aphid resistant soybean plant of claim 26, wherein said linked polymorphic loci comprising alleles or combinations of alleles that are not found in a aphid resistant soybean varieties harboring said aphid resistance locus comprise alleles of at least one marker selected from the group consisting of NGMAX007665590, NGMAX007665668, NGMAX007666264, NGMAX007666309, NGMAX007666777, NGMAX007666843, NGMAX007666869, NGMAX007666919, NGMAX007666921, NGMAX007666976, NGMAX007666977, NGMAX007667014, NGMAX007667071, NGMAX007667072, NGMAX007667077, NGMAX007667093, NGMAX007667095, NGMAX008369615, NGMAX007667202, and NGMAX007668494 and/or comprise alleles of at least one marker selected from the group consisting of NGMAX007669116, NGMAX007668903, and NGMAX007668494.

32. (canceled)


SEQUENCE LISTING (82 sequences)

SEQ ID NO: 1 - DNA, 201 bp, Glycine max
gtccagtacatacgcgtttccaagatatgcttttcatttataaaaatagcaaacttacagttgtttgtaatatgcaggctttaatatattgtcattgaaahtagtctctacttaaattgtgtttttaatttctgaaattacatagaaaacatattcctagttattgacaggggactaaaactaaattatatatacacacac

SEQ ID NO: 2 - DNA, 201 bp, Glycine max
ggaaatctatgtgggcacgaacatcttcctttactcatttacaccattcctatttgggaactgattttttttttttaaaaaaaaagctgaatcaggcaaahtttgaaaatgatttttcagttttacttgtgtttgtttgagattttacgtttgtccctaatatggtcgttttatgtaacactcaattgtttgaatttatta

SEQ ID NO: 3 - DNA, 201 bp, Glycine max
tttactcatttacaccattcctatttgggaactgattttttttttttaaaaaaaaagctgaatcaggcaaattttgaaaatgatttttcagttttacttghgtttgtttgagattttacgtttgtccctaatatggtcgttttatgtaacactcaattgtttgaatttattatggcgtaatggttcaaccaataacgtcct

SEQ ID NO: 4 - DNA, 201 bp, Glycine max
acatttaacaactttttaacaagtcgaacttatttaagttagtcaagcataattcaatgaaagataataagatccttgtgttatgatttttggagttagthgtagataatattgtgatggtgatttaaagattacaacataaattggaccatgtaaataaatagattttagcctatgttaaatcttcaaaacaatttctct

SEQ ID NO: 5 - DNA, 201 bp, Glycine max
ctttttaacaagtcgaacttatttaagttagtcaagcataattcaatgaaagataataagatccttgtgttatgatttttggagttagtagtagataatadtgtgatggtgatttaaagattacaacataaattggaccatgtaaataaatagattttagcctatgttaaatcttcaaaacaatttctctttatttttata

SEQ ID NO: 6 - DNA, 1252 bp, Glycine max
  misc_feature (240)..(240): n is a, c, g, or t
ggccagcttgcatgcctgcaggagaagtatcgcaaaacttaagagtgaaggaaaaagcagtgttgttcagtttttcaccttctcttattttaagcctagctttcacataattaacttttctttccaaaactcaagtttahgatttgaaaaactatgtcctctttgatgctttataagaccatatagattttcttgcctagaacagtggtcaataatttggaactatagtgactttgctgntgtataaatttatatttaatatagaattatcaattttcttattgcatctcaaatctcaatgcctacctattcctcctcatgcaggacatgaagcggcaatgtgatgaaaaaaggtttgtatttacacaactgctattgttttttctcatcattatgtgatgtttcctgaatctgttttattgccttcagagatgtttatgaatacatgattgcccaacagaaagagaaaggaaagtcaaaaagtgctaaaggtgaaagtttcacgctacagcagttgcaagcagctcatgctgaatatgaagacgaagcaaaactttgtgcctttcggttaaaatcgctgaagcaaggccagtcacgcagtctcctaacacaagcagcgcgtcaccatgctgctcaggttttcttgaactatgacctttctcattcaaatcttttatcatttcttcagcctagtcttgatgcatctttgttcttgttcttctttcaatatagttgaatttcttccggaaaggacttaaatcactagaggctgttgacccacatgttaggatgattgctgaacgacaacatattgattaccaattcagtggcctggaggatgatggaggtgaaaatgataataatgatgatgggaatgattttgacgtcattgaaggtggtgagttgagttttgactacagggcaaataagcaagggccatatattgtttccacatcaccgaactcagcagaggtaggaaatttgtattactcaaaacttgaatggttttcaaagcctgggtgcatattgaactttattcttattgctcatttgcttttttatttaaaatattaccaaactttgtcacagttgtcattttatacttgttgcagtctatagctcgctacaaattaaaaacattccattgttgttttcataatctgaactataaattcatcttatcataatcattggcaatgttttgccaggtggaagaatcaggccgttcatatattcgagcttcaaccccaga

SEQ ID NO: 7 - DNA, 201 bp, Glycine max
aatactatccggagaaagtttgatagagaacttatcactaagcattctcaaaccctttttttaatctaaaaacaaacttttatttatcttgtactgtagadattcttaatctagaggaaagttttatttgtaataattaagtttgttaaaaaatgaagatgagataaggaaccaatttttttttctctttggtcatcataa

SEQ ID NO: 8 - DNA, 201 bp, Glycine max
tttaactactcattattttacctttaaaattttgtgaacatatttatttaactttcccattttctcctctcttatttcctatttgactttattaaatgatbgttccaaggtaaacacgccatagttaaccttcttataatatatgtgggactcaatcaagtgggatcaataacactaaggatacaaattgtatcttaggaa

SEQ ID NO: 9 - DNA, 201 bp, Glycine max
tttgatgcctcctctctcatgtggctgaagccattgcttttgggtcaagttattttttaatatgattgccaaagattagtacaagcatggaagaaacaagbtagctgtgggtgataatagatattttgaaggaattgtttctgattgcctgcttttggtagattacaattcccagcaagatcttgtttttgcccatagaac

SEQ ID NO: 10 - DNA, 201 bp, Glycine max
ttttgtatctgggaaaaaaaacattcaatcagggatgcatgcattgtctcacacactgacacataacgatggcatccatggtgttgctgcagcctgcagchgagctggatttacacaaaaatgagacctagatgaaacgggtttcttataccaaaaatgaggcaccaatgctacaaagtacaaacaaactaatatattgca

SEQ ID NO: 11 - DNA, 201 bp, Glycine max
gtccatttgtatgtgtctgttcactagtaatggctgcagttgggggtattgtatctattcagtgaaaagaaagttattagcaaaaccccaaaatggtttabtgaagtttagtattatggccagagagaatctggcttaaatggggtgaggaggaaaacaaattaagctaatgccatttctcttcattcttcaattttctag

SEQ ID NO: 12 - DNA, 201 bp, Glycine max
cttcatgggtattggtatagtccctcatggcctcttgttcatcgttttttctattttttaataatactttgggctagagtaaaggattgataggatgtggdgtgtcatcatagttgggatgtcattgttctgttttaatgctttttaggccataattttcattgaaaaaatatacacgcgggtgtgcacatacccatgcac

SEQ ID NO: 13 - DNA, 201 bp, Glycine max
tatatatattgacaaaatagaggattgaaccaaaattgtgaattggaaaaacaacgagaatcaaaattatatttaagcctttatctgttcaaatgttaaghatgttcgaccgtgtgatagagagttgacactatgatactctactcttaagaaaataccataaaaactatataagaaacctacatagaagataaaataaca

SEQ ID NO: 14 - DNA, 201 bp, Glycine max
taaaaactatataagaaacctacatagaagataaaataacaactcatatatttttattgatgctaatagataaaggaaaatacatttaaatagtcctaaahgatattaattcacagataatacttatccaggtgtcaacatcatgttattctaaacatatttaaactaaaattataagaaaaaatatttaactccacccaa

SEQ ID NO: 15 - DNA, 201 bp, Glycine max
aagtgatctagggtagtgatatagtaggaaacataccgacacgatcaagaaagctaagagaggaagatacacacaattcctcaatctcaatgctctcatchttccaaatgcaaatgatggttttatggcctttaggcctaacttaatcccagcctgaaagttaggtcttgtgtttatttaagtgctattgaaaaagaagcc

SEQ ID NO: 16 - DNA, 201 bp, Glycine max
agtcacgcatgatatgcattcttcactgtaaacaaattatcattcgtatgtttccaagctggacaatcctaattaaaaaataaaaatagaaaaaagaatcgbaagtaattaacaaccctaattagtccaagataaaaaaaagagatggaatgtaaattttttttgtcagattcgtgtcattgttacagttttcaaagtgaa

SEQ ID NO: 17 - DNA, 201 bp, Glycine max
tgcattcttcactgtaaacaaattatcattcgtatgtttccaagctggacaatcctaattaaaaaataaaaatagaaaaaagaatcgaaagtaattaacahccctaattagtccaagataaaaaaaagagatggaatgtaaattttttttgtcagattcgtgtcattgttacagttttcaaagtgaaaaataactaattta

SEQ ID NO: 18 - DNA, 201 bp, Glycine max
aactcgtgcgtgcaccccgaacacctgaaaaagcaataataatataaataaattgaatttttaaaaaggaaaaaaaaatgagagtaagtaaaggaggagcdtacgattggaagtgcggacagtgaagtgcgcatgttagcggcggggactgtggaattgaagaaataagcgtcggcgccggcgtcggcgtcggtgtcggcg

SEQ ID NO: 19 - DNA, 201 bp, Glycine max
taagcgtcggcgccggcgtcggcgtcggtgtcggcggagggagggcggtggatgcggtggcgcggcaggagaagagagagaacgacggcggagctgacacbagacggagtgtcatttttccggtacgacgtgggaatggataataattacaaattatctttataagtagttcctataatatctgtagcggagttgtgggaa

SEQ ID NO: 20 - DNA, 201 bp, Glycine max
cactcacgatgagtgatgctaactgctatttgttttggttgggtcatttcctactacatcattcgcagagaagagcgaatgacatttaaaaatcaaatgavccgaatgattcgatcggtcagtactcagtatgactgtatgagttggctaaatctcttatccaattttcatagtttcattatttcatttaattatggttta

SEQ ID NO: 21 - DNA, 201 bp, Glycine max
ctcccccggcgcagctcagcttacatgcattatcaggccattcagcgcctgaaaccttacgtctgaagggggccattaatgagcttcacgttaatatccthattgatggaggtagtacgcacaacttcctccaccacagggttgcgacggtgttaggtctttcacccaaagagatagctccacttagagttacggtgggta

SEQ ID NO: 22 - DNA, 201 bp, Glycine max
gcttcacgttaatatccttattgatggaggtagtacgcacaacttcctccaccacagggttgcgacggtgttaggtctttcacccaaagagatagctccahttagagttacggtgggtaacggagacgaaatccgttgtcatcagctttgcatggctgttaaagtacaaatccaaaggtactttttcacggttgactttca

SEQ ID NO: 23 - DNA, 201 bp, Glycine max
taacggagacgaaatccgttgtcatcagctttgcatggctgttaaagtacaaatccaaaggtactttttcacggttgactttcacatcttaccattgtgtdgcgcgtacgtcgtactaggagtagagtggctcaaaacactggctccaatgctcacagattacacttcattgaccatgaagttcattactaaaggcaagct

SEQ ID NO: 24 - DNA, 201 bp, Glycine max
gacgaaatccgttgtcatcagctttgcatggctgttaaagtacaaatccaaaggtactttttcacggttgactttcacatcttaccattgtgtggcgcgtdcgtcgtactaggagtagagtggctcaaaacactggctccaatgctcacagattacacttcattgaccatgaagttcattactaaaggcaagctagttgaa

SEQ ID NO: 25 - DNA, 201 bp, Glycine max
aatatggcttccctctaatttgtctttcattaagttgttactcgaagaatttcatcaatcacctgcaggcgcccatatgggagtgcaaaagaccttacathgtctacaggaaaacttcacttggagttcaattcgagagtatacacgtgcttttatcgcaagctgcttaacctgtcaatacacaaagtacgataaccggaa

SEQ ID NO: 26 - DNA, 201 bp, Glycine max
tcacttggagttcaattcgagagtatacacgtgcttttatcgcaagctgcttaacctgtcaatacacaaagtacgataaccggaaaccagggggcttgcthtgtcctcttccggtatcggcacaaccctgggaagatttgtcgatggactttatcgtagggttgccaacttatcggggaaatacttgcatctttgtcgtgg

SEQ ID NO: 27 - DNA, 201 bp, Glycine max
cattattaatatgtaaagcaatacaaggaggacaataccggagcagcatgtcttaaactttcttttgttcacctattagtctattacggagttatatatabtagtatcacaatgtataacaagtaacaacagatatcaaactacaacaatagtgcctcaattggtcatatgcaaaacttattgaacatgcttaagagtatt

SEQ ID NO: 28 - DNA, 201 bp, Glycine max
actcaatacaattcattattaatatgtaaagcaatacaaggaggacaataccggagcagcatgtcttaaactttcttttgttcacctattagtctattacbgagttatatatactagtatcacaatgtataacaagtaacaacagatatcaaactacaacaatagtgcctcaattggtcatatgcaaaacttattgaacat

SEQ ID NO: 29 - DNA, 201 bp, Glycine max
gttatatatactagtatcacaatgtataacaagtaacaacagatatcaaactacaacaatagtgcctcaattggtcatatgcaaaacttattgaacatgchtaagagtattcaaatccaaaagtcagcaattataatgatgcgccccaattataaatttttttgaaataatttaagtcccgtaagaaaaatagttaattta

SEQ ID NO: 30 - DNA, 201 bp, Glycine max
agacgaaaaaataatttctattttctctactatttagtatgacaatcaagaatcaaattatcactatattttttcttttttctacttcttctttcaagtghagaaaaaccctaaggagttgcggggttttttctaccaattggggtcctcccttcaccactcgcatgcggatggtttacaagattcataactattcttctt

SEQ ID NO: 31 - DNA, 201 bp, Glycine max
actatattttttcttttttctacttcttctttcaagtgcagaaaaaccctaaggagttgcggggttttttctaccaattggggtcctcccttcaccactcbcatgcggatggtttacaagattcataactattcttcttattagaggacacttacctaatcaacatttaaatttggttctacctaaatttttttggtttac

SEQ ID NO: 32 - DNA, 201 bp, Glycine max
caccactcgcatgcggatggtttacaagattcataactattcttcttattagaggacacttacctaatcaacatttaaatttggttctacctaaattttthtggtttactccaacatttcaaaaaaaatatcaacaacacatttagaggacacttacctagtcaattattttttcctatgtttttttagcttattttaata

SEQ ID NO: 33 - DNA, 201 bp, Glycine max
gcatgcggatggtttacaagattcataactattcttcttattagaggacacttacctaatcaacatttaaatttggttctacctaaatttttttggtttahtccaacatttcaaaaaaaatatcaacaacacatttagaggacacttacctagtcaattattttttcctatgtttttttagcttattttaatactactaac

SEQ ID NO: 34 - DNA, 201 bp, Glycine max
acatttaacaactttttaacaagtcgaacttatttaagttagtcaagcataattcaatgaaagataataagatccttgtgttatgatttttggagttagtdgtagataatattgtgatggtgatttaaagattacaacataaattggaccatgtaaataaatagattttagcctatgttaaatcttcaaaacaatttctct

SEQ ID NO: 35 - DNA, 389 bp, Glycine max
aaaagtgtatacatgaggattgagggtacatatatttagggatgatttaccccatgtacatgtggatcccaattgctaacatgaagatgcacacgagagchaagtagaggatagcatggatcaatattgataatccactggtctgaaaattaccaaactctatgaacctgcctcttccaggtatttggaccaagagccctggagtgaggagaatgaaaagcaccacagacacaaaaactggcccccaatctcccatttcctccttctccttaatttctctttgatctcctaattatttctctatctagctatgtgaatggtatagtagttgcaatgacaatgagtgcaggaggacttaaaaaggaatggagctgaaaagtggtcacttt

SEQ ID NO: 36 - DNA, 201 bp, Glycine max
accttcaaaaatatgaaatattcttcagtaggaacagtcttcaaagctcccacaattttgtcggagccaatggtgtctagattgatgctattgaagtattdgtttcttaaatgttgcataatttcttaaatgtagcactaatgttgcaatcttcagggagcaacaacatttttggtgctggttgtgcatacgaaagagtaa

SEQ ID NO: 37 - DNA, 201 bp, Glycine max
gtagcactaatgttgcaatcttcagggagcaacaacatttttggtgctggttgtgcatacgaaagagtaatggagattttgttttggttgaaacatcatgdaagcatgtttccccactagtgttcgagggagaaacaataactttagtggatgtcatcacttggttcaaagaaattaatctacatgttattatggaatttg

SEQ ID NO: 38 - DNA, 201 bp, Glycine max
tgcatacgaaagagtaatggagattttgttttggttgaaacatcatggaagcatgtttccccactagtgttcgagggagaaacaataactttagtggatghcatcacttggttcaaagaaattaatctacatgttattatggaatttgattgcaagcacattgttgagtgtttagcaaatgatagcacaaatcacattgaa

SEQ ID NO: 39 - DNA, 201 bp, Glycine max; identical to SEQ ID NO: 37

SEQ ID NO: 40 - DNA, 201 bp, Glycine max
attcaaataacagagtggttttcagtagtttctgtagtggctgcaactgaggaaaaatacatgtcacaccaacatattgtggccataccctgtagatctchaaatagagtagtagagttccatagaaaaacattctgtaattgtttgattaaaagataggcttaaatacttagtccttgcaatttagctttttttttcatc

SEQ ID NO: 41 - DNA, 201 bp, Glycine max
tgccatgttattaatctcaaacaatgatacctattattatttgagtgatcaattccaatctatccaaatttcttaatttggaagatttcactgcataacavtctcaaaagtgtattcttaatttggaagatttcactgcatatatacagatttttcacctaaaaaggtgtcttattagtaatatttttcttattaaaaaag

SEQ ID NO: 42 - DNA, 597 bp, Glycine max
  misc_feature (395)..(395): n is a, c, g, or t
gtttttcctcactctctccatcatgttcatgtcaccactctccaagtagttactcccttgcahgtcatgctaacttggagagctgattgcatgcttctctgtaacataatcctagtgtacaccttaataggatgggtttcaattattcagttgttgaaaagtcattactactcagctaggaaaggcaggcatggaatggccattttctaaataatttgttataacaattgaagagagtgataacagggtaagaagtgagtgaaagctacagcctacacaaaagagagaacttactttgaaaggaatttataaaattgaatcaccaaatccaggtcattctcatataccgtactgagttttcccaggcatgagatgccaaatcttgggtctgttgnataaattatattaataacaatgtttcagaataaaatactatgaagtttggttatacaaatacaatagaacagattctgcatgcaaccattccatgtatcaaaatgctcaagttaaccccacagctatcctagactatataatgggaggaaaagaaatgtanagtaaaataaaagttaagaaaggtcattcctttcaaatgta

SEQ ID NO: 43 - DNA, 201 bp, Glycine max
caacaaagcatttgcttctgaagcaatctcagaagaagtagcaaatgcacagaagtacatcacaagaagcagcgaaagtggttggagcttcatggacagadgagttggaaatgtgaacaccatgaggaattttttaggaaagttttgatcaccgattgtctgatacaatcaaatgaataggagaagtacttgcatgcaatt

SEQ ID NO: 44 - DNA, 201 bp, Glycine max
ctcatctaagtaaaccttatcaagatgtactttcttttcctcgagaacatctcagatccaatgataccttacgtcaaggtgcacatccttgaacatgtcthcatcatgagcgatgttatcctctatcttgaactgcacgagctagtgacggctccggatgaacctgacttttaataccactgttgggaaaaactcgatggg

SEQ ID NO: 45 - DNA, 201 bp, Glycine max
tactacgttggtattgtcataagcacgacgtagaaagcacgggtttcaactacggtactaacctaagcacgacatagaaagaacaagtattgtcatcgtcvctcatgtcaacaacgtcttcgaaagcttacgttcaattactgtcgtggctgaccccgtcgcaggattcagtgctttctaaaacggtgtgttgcgaccgtc

SEQ ID NO: 46 - DNA, 201 bp, Glycine max
ccttaagaaggattctcaaaagtttacttttagctccaacaagacatgttcttacatctaagcccaaccaaacaaaaatagaaaaaccaaattttaaatthtttattatcaacctcatgatcaccatgtctaccacgatttatccatggttgtgtttggttatcaattttagctttttcatcaattttggttaataatttt

SEQ ID NO: 47 - DNA, 201 bp, Glycine max
aagtttacttttagctccaacaagacatgttcttacatctaagcccaaccaaacaaaaatagaaaaaccaaattttaaattttttattatcaacctcatgvtcaccatgtctaccacgatttatccatggttgtgtttggttatcaattttagctttttcatcaattttggttaataattttgattcaaattcaatcgtca

SEQ ID NO: 48 - DNA, 201 bp, Glycine max
tattttgttataagattttatctcatacttatactttaataaaaaaaatattaaaaataattaatcaaatttcatgatagatattaatcatcatcaaactvgataagtagaattggttcactaacccaattaaaaacaggccctatgatattgccccgttaagaaaaatcaatgggggccaaatagtattgtgttttttaa

SEQ ID NO: 49 - DNA, 201 bp, Glycine max
tagattacttcttgattattgaagtagagtatcttatggatggttattatcccaagccaattatttgtgatacttacttgacaagggtcacatgattgagbctaaagcttttacacttttgtgattggggagtgcaagctctccaagtgttgtgttgattacatggaaattcctagggatgacaaaaaaattgaagatatc

SEQ ID NO: 50 - DNA, 201 bp, Glycine max
ggattgcttttatgtaattatagtaattaggcatattttgcaaatccttaattatgactccctctataggtaacatcttttggtgagtgattcacatacahgaatggttccctcgatgtcttttcttaacaattattgaattacttaatgtgaagtgtgaattttaacaaattaattatagtcgtgaaatattttcaagtt

SEQ ID NO: 51 - DNA, 201 bp, Glycine max
tgaagttggcagcctccttggcaggatcatccttagagtttgaggcaacaaaaccacgcttgagatccttgactgcaacattcttcacattaaatcctacvttgtcacctggaagggcctcctggagagcttcatggtgcatctccacagacttaacttcagttgtcaggccagtgggagcaaaagtcaccaccataccgg

SEQ ID NO: 52 - DNA, 201 bp, Glycine max
agtttcccgatcaactctactataaaaagaggttcatgtataaccaaatgttgttggatccttaatcctacagtttaggtaaacaattatgcattgtgacdgtcatttaagattaagcccaattaaaagtgatctaataaggtttaagaaatttgggctttgatgtatctaatagggtataaacctttatgtatagagacc

SEQ ID NO: 53 - DNA, 201 bp, Glycine max
gacatcccatgagtagagaaaggttgagagaaaaactaactcaagagagtacgtgtcaacctaaaagaccaaattgcacaacttcaaagagcgattgatcvtcatgaattcaggtatgctttcatttcaatttcttaatggttaaaatgattgattcgagattgatagatgacagatttaatttaagggagcgtttggtag

SEQ ID NO: 54 - DNA, 283 bp, Glycine max
acggaaaataaatgaaactaagaaaataaaattgaaattttatgacaactaaaaacaaaataataataataataataataataataataataataataatatacaatagaagccaaaccatactccaaaacaagaaaacatcaattagagtaattgttagttaatgcagttagcttagtcaacaaaaagctcctgttcctgtaaccaaaatatataccctgcctaaagacaacataacatagacacttgaatcaaattcttctgcttctctatctgatagagt

SEQ ID NO: 55 - DNA, 890 bp, Glycine max
  misc_feature (617)..(617): n is a, c, g, or t
attttaccctgaggttttgccatatggtgaaactttcttccatgagccagttggtagagcttctgatggtcgccttatcatagatttcattggtatataccttttcaataattctcatcatttttttgctttatttcttattcatttatttgtgaaacacgatcaaatgacatgtttgctttttctatctactttgttttctttttctttaatcatcatgtggtgtctcacaacacaagctaaatcttcttttctttgagttttgttccacaaagattggattccagtttgtgttgttattggagttataagatacatgtggttgaagggagaagagggaagggtgatggaggtcatgagttagaatcttttttattaacaaaaattaacaaattaacaactaatatatattgachgataaaaagaattgtgttgtcattgactaagtgaccaacacaaacatctctacccaaatgaataaagttgggattcaataagaatgagttgtgaaatttttttacacaatatctctgtctaccattatctatttagcagccatagaatcctatgatggtcatacttacaaagtggtacataaaaaatataaagttttcacnaagtgaaaatttaatatgagaagatcataatatatatattaagggatctagttcaattngttgaagtgtatataagtattgtaaatttcctcttatcgtgttcaatttctgcatatcaaaaaatatatcataatatatatagttctattgccaagaaactatcatactagtgatttggtgccatttgtgtttgcagcccagcatctaggtttcccattcttgagtgcatacataaattcaattgggacaagttataggcacggtgcaaacttt

SEQ ID NO: 56 - DNA, 614 bp, Glycine max
  misc_feature (399)..(400): n is a, c, g, or t
atggatgttatcaataagtgcttctctaaaaaggctgttgaggaaatattatcttctctcgtaagttgtattgagttacactattgaaaatgtctgattahtattcttcagcatgatcaaggatgaacttttgccatttagcaacctgaattttagcctgctgttttaggaggtggaagctacgagaaaggctgatccttggatttctgcaactattcaatccttaaagaaagcatctccaacaagccttaaaatctttcttagatcggtatgtgtccagaatgtattaaaagttctgttttatgtacattctaaaattcacttctcttctgtgttcaactgttgaagtatcatttttatcatcatcatcattttactatcattattgtgattataacnnctgttttttttttaaatttattttggcctttttcagattagacaaggaaggctccaaggtgttggacaatgccttgtttctgattatagagttgtttgtcatattctaaaaggacactacagcaaggatttcttcgaggttatctgatgattcctatacatagttatgcttttaattatttttctttcatgccctatggttaatgttacccttt

SEQ ID NO: 57 - DNA, 706 bp, Glycine max
aggtgatgcctttggaatttttgcttgttctttcttttctcttttaatattttactttattcactgttcttaatagttccatgccttgtttatgtataacttttctattttcagttgctggttttggttgtttaagtaaggttggacattagacatttcaaaattgaaaccaatactctgatgcattacatagcagttcctgattcatcgcatgttttaaaaagtaatttatggtgtgtcaaagccatcatttttctatttttatgcattgtcgaagaatatgtggattcgacttgtcttatactgtagttaagaacatttgttgttgtctaaataagatgtatatgctgttgtgcttcttaaagcagcgtatgttatgcgttagtagaaggtacaaatggctgaacgggcaaaacctattattgttttgtttatgatgagaaatgagaaataaatgatgctgcttggtgctattttgttattctttccattgtatttgataaacataaacagtaaatatgtaaaacatgatactgtaatttcttcaattgatggatghagtggactgcattctcatgtatagaagctgatatttgtttttttgtcttgggatgtaatttgcaggagggtgctgctgctgttgctgtggaagaagccaaaaagagtaatcatctgcaggtcgactctagagataaccccgggtacc

SEQ ID NO: 58 - DNA, 441 bp, Glycine max
taaacatgtgacaatatgaaattaattaaattttatgcaatgaaaaagaaattttaaaatattttgtactttgaagcaaattaaatacactaaacgtgtttattttttaagaaatttgacacaaattaaccaaataagcaaatattagacatggaagatatttaataaaaaataaaaaaaatcaagtagtcatgaactcaacatccaaaacaacttatacatgaacatgcatgctgccttgaaggcataacatatcaaaaacatggctgtcatatatataaggttaataagtttatataaacaacagaataggaagtttcacttgdatgagaaccattgggcatattagatggtagtgtttcctgcttaataggtgattgaatatcatttctaaaagtatgagtttgccatatgaactaaaagggtctctgatacctgtac

SEQ ID NO: 59 - DNA, 453 bp, Glycine max
  misc_feature (270)..(270): n is a, c, g, or t
gcatgccttgcagaaatttaagaataaaagtttaaacatttttgtaaccaaatttcaacataaaactaaaaaaaattagaagcaaccacatgttgcaatttaaagttaaaaatdgtacagagatctattccctaccacaatgtatctgtcttcagtaacagctagcatttcccaccacgtgatgaagccttttaatggcttccttcatctccttcatttcccaaatattagacatggaggatatttagaaaataaataaaaataaagtantcaagctgtatgataagtaattatttatatgtaattactttctttcccaaattaattcgatactagtaatctttacttaaaaggctttaataaatatataataattaataaaaatatatatacttattaataaaatatactgtgagtaataatacaatcttaagaatattatctaaggtagaa

SEQ ID NO: 60 - DNA, 170 bp, Glycine max
tctctaacaaagggcaagggataaacaacaaatggcacaacatagacggcgacaaagccgatgacaacgacagagacaaaggttccggtggagagaatggcaccgaagccgatgcggcattggtcggcgcggagggcaatgagggggaagatgtcgagggtgctgttgcc

SEQ ID NO: 61 - DNA, 606 bp, Glycine max
agcctgacctacatgttggtgtggttggtctcvgtggactaggtcacatggctgtcaagtttgccaaagcttttggagctaaggtcatagtaataagtacatcacctagtaaaaaggatgaagcaatacaacatcttggagctgattcctttctactaaaccgtgaccaagatcagatgcaggcaactatatatatgccaatgccatgcatttggaatattgtgaaaatatcaaaatacacacatttctctcaattaactaatgttgataatttcgtagggtacaatgggtgctttggatggtattattgacacagtttctgcggttcatcctctcttacctctcattggtttgctcaagtctcatggaaagattgtaatggtggatgcaccagagaggcctctagaactaccagtctttcctttacttgctggtaagcatgtgaaaaatatttccctttataggatgcagagcacatgaaagcatatgtgatgattcatatataagaatagtttagtgcaaaacttaagtagattgatgccaattaacttaggtactatttaacataattggcaaggagaaagatagttgctggcactctgattg

SEQ ID NO: 62 - DNA, 210 bp, Glycine max
gcacaatgacaatcacatacaatgagcgaaattagtttctgacccagtgtgttagggacatctgtgatctctgatccagaatcaatttttttccactcaatattaaaaaaatgaaaagtgcattaagaaacattattattattattattattattattattattattattttcgtcctgataatttatccttttttatgccttaatgtga

SEQ ID NO: 63 - DNA, 26 bp, Glycine max
tgtttccaagctggacaatcctaatt

SEQ ID NO: 64 - DNA, 27 bp, Glycine max
ggtcttataaagcatcaaagaggacat

SEQ ID NO: 65 - DNA, 20 bp, Glycine max
actcaagtttaagatttgaa

SEQ ID NO: 66 - DNA, 19 bp, Glycine max
aactcaagtttatgatttg

SEQ ID NO: 67 - DNA, 32 bp, Glycine max
gtttccaagctggacaatcctaattaaaaaat

SEQ ID NO: 68 - DNA, 25 bp, Glycine max
accacttttcagctccattcctttt

SEQ ID NO: 69 - DNA, 15 bp, Glycine max
cctcctgcactcatt

SEQ ID NO: 70 - DNA, 15 bp, Glycine max
cctcctccactcatt

SEQ ID NO: 71 - DNA, 25 bp, Glycine max
tgttcatgtcaccactctccaagta

SEQ ID NO: 72 - DNA, 20 bp, Glycine max
gcatgcaatcagctctccaa

SEQ ID NO: 73 - DNA, 16 bp, Glycine max
cttgcaagtcatgcta

SEQ ID NO: 74 - DNA, 15 bp, Glycine max
tcccttgcatgtcat

SEQ ID NO: 75 - DNA, 25 bp, Glycine max
ggagcagcatgtcttaaactttctt

SEQ ID NO: 76 - DNA, 36 bp, Glycine max
agtttgatatctgttgttacttgttatacattgtga

SEQ ID NO: 77 - DNA, 23 bp, Glycine max
ctattagtctattacggagttat

SEQ ID NO: 78 - DNA, 24 bp, Glycine max
cctattagtctattactgagttat

SEQ ID NO: 79 - DNA, 30 bp, Glycine max
cctccttctccttaatttctctttgatctc

SEQ ID NO: 80 - DNA, 25 bp, Glycine max
accacttttcagctccattcctttt

SEQ ID NO: 81 - DNA, 15 bp, Glycine max
cctcctgcactcatt

SEQ ID NO: 82 - DNA, 15 bp, Glycine max
cctcctccactcatt